(19) United States
(12) Patent Application Publication
(10) Pub. No.: US 2026/0187402 A1
(43) Pub. Date: Jul. 2, 2026

(54) SAFETY FEATURES FOR A PRESCRIPTION-CONTROLLED, METERED-DOSE, AEROSOL INHALER DEVICE

(71) Applicant: MicromistRx Inc., Orem, UT (US)

(72) Inventor: James Kevin Shurtleff, Orem, UT (US)

(21) Appl. No.: 19/439,176

(22) Filed: Jan. 2, 2026

Related U.S. Application Data
(60) Provisional application No. 63/741,259, filed on Jan. 2, 2025.
(60) Provisional application No. 63/791,864, filed on Apr. 21, 2025.

(51) Int. Cl. G06K 19/07; A61M 15/00; G06K 7/10

(52) U.S. Cl. CPC G06K 19/0723; A61M 15/0001; A61M 15/009; G06K 7/10297; A61M 2205/276; A61M 2205/3306; A61M 2205/3584; A61M 2205/505; A61M 2205/6009; A61M 2205/6054; A61M 2205/609

(57) ABSTRACT

An inhaler apparatus includes a receiver port of a dispenser. The receiver port receives a canister with an inhalation substance. The dispenser dispenses the inhalation substance from the canister. The apparatus includes a wireless data module that reads encrypted data from and writes encrypted data to a memory chip affixed to the canister and a canister code module that, in response to data read from the memory chip not including a cannister code, writes a cannister code of the dispenser to the memory chip. The apparatus includes a dispenser module that enables dispensing the inhalation substance from the canister in response to determining that the memory chip includes the cannister code and that locks out the dispenser from dispensing the inhalation substance from the canister in response to determining that the canister does not have a cannister code or determining that the canister includes a different cannister code.

### CROSS-REFERENCES TO RELATED APPLICATIONS

This application claims the benefit of U.S. Provisional Patent Application No. 63/741,259 entitled “SAFETY FEATURES FOR A PRESCRIPTION-CONTROLLED, METERED-DOSE, AEROSOL, INHALER DEVICE” and filed on Jan. 2, 2025 for James Kevin Shurtleff, and United States Provisional Patent Application Number 63/791,864 entitled “ADDITIONAL SAFETY FEATURES FOR PRESCRIPTION-CONTROLLED, METERED-DOSE, AEROSOL, INHALER DEVICE” and filed on Apr. 21, 2025 for James Kevin Shurtleff, which are incorporated herein by reference.

### FIELD

This invention relates to aerosol inhalers and more particularly relates to safety features for a prescription-controlled, metered-dose, aerosol inhaler device.

### BACKGROUND

Current inhalation devices, including propellant aerosolization, piezoelectric mesh aerosolization, compressed gas aerosolization, thermal vaporization (vape pens or devices) have attempted to control the inhalation process. However, they have all omitted one or more of the following key elements for a safe, metered dose, controlled inhalation process: 1) the containers or cartridges with the liquid formula to be aerosolized are not tamper-resistant, which means they can be opened and the contents used in unsafe or improper ways, 2) the prescription information saved on the near field communication (“NFC”) tag on each cartridge is not encrypted to prevent modification, 3) the cartridges are not configured to prevent their use outside of the specific control device for which they are manufactured, and 4) the drug containing cartridges can't be exchanged without losing the prescribed dose time.

### SUMMARY

An inhaler apparatus for delivering a prescription-controlled, metered-dose, aerosol includes a receiver port configured to receive a keyed canister with an inhalation substance. The receiver port includes a key shape to receive a keyed receiver top of the keyed canister and to reject canisters without the keyed receiver top. The inhaler apparatus includes a wireless data module configured to read encrypted data from and write encrypted data to a memory chip affixed to a side of the keyed canister locked into the receiver port, a lockout device configured to prevent, in a locked state, the keyed canister from dispensing a metered dose of the inhalation substance while in a locked position, and a dispenser module configured to maintain the lockout device in the locked state, to read data from the memory chip that includes information including timing for a next metered dose of the inhalation substance, to unlock the lockout device at a time for the next metered dose of the inhalation substance, to lock the lockout device in the locked state in response to a user dispensing the next metered dose, and to signal the wireless data module to write encrypted data to the memory chip comprising a time of dispensing the metered dose. At least a portion of said modules include one or more of hardware circuits, programmable hardware devices and executable code where the executable code is stored on one or more computer readable storage media.

Another inhaler apparatus for delivering a prescription-controlled, metered-dose, aerosol includes a receiver port configured to receive a keyed canister with an inhalation substance. The receiver port includes a key shape to receive a keyed receiver top of the keyed canister and to reject canisters without the keyed receiver top. The keyed receiver top of the keyed canister includes a shape configured to mate with the keyed shape of the receiver port during insertion of the keyed canister and to lock into place when the keyed canister is fully inserted. The inhaler apparatus includes a wireless data module configured to read encrypted data from and write encrypted data to a memory chip affixed to a side of the keyed canister locked into the receiver port, an encryption module configured to decrypt encrypted data read from the memory chip and to encrypt data to be written to the memory chip, and a lockout device configured to prevent, in a locked state, the keyed canister from dispensing a metered dose of the inhalation substance while in a locked position. The inhaler apparatus includes a dispenser module configured to maintain the lockout device in the locked state, to read data from the memory chip with information including timing for a next metered dose of the inhalation substance, to unlock the lockout device at a time for the next metered dose of the inhalation substance, to lock the lockout device in the locked state in response to a user dispensing the next metered dose, and to signal the wireless data module to write encrypted data to the memory chip comprising a time of dispensing the metered dose. At least a portion of said modules include one or more of hardware circuits, programmable hardware devices and executable code. The executable code is stored on one or more computer readable storage media.

A method for operation of an inhaler apparatus includes receiving, via a receiver port, a keyed canister with an inhalation substance. The receiver port includes a key shape to receive a keyed receiver top of the keyed canister and to reject canisters without the keyed receiver top. The method includes reading, via a wireless data module, encrypted data from and writing encrypted data to a memory chip affixed to a side of the keyed canister locked into the receiver port. The method includes preventing, using a lockout device in a locked state, the keyed canister from dispensing a metered dose of the inhalation substance while in a locked position, maintaining the lockout device in the locked state, reading data from the memory chip that includes information about timing for a next metered dose of the inhalation substance, unlocking the lockout device at a time for the next metered dose of the inhalation substance, locking the lockout device in the locked state in response to a user dispensing the next metered dose, and signaling the wireless data module to write encrypted data to the memory chip comprising a time of dispensing the metered dose.

Another inhaler apparatus for delivering a prescription-controlled, metered-dose, aerosol includes a receiver port of a dispenser. The receiver port is configured to receive a canister with an inhalation substance. The dispenser is configured to dispense the inhalation substance from the canister. The inhaler apparatus includes a wireless data module configured to read encrypted data from and write encrypted data to a memory chip affixed to the canister and a canister code module configured, in response to data read from the memory chip not including a cannister code, to write a cannister code to the memory chip on the canister. The cannister code is specific to the dispenser. The inhaler apparatus includes a dispenser module configured to enable the dispenser to dispense the inhalation substance from the canister in response to determining that the memory chip includes the cannister code and configured to lock out the dispenser from dispensing the inhalation substance from the canister in response to one of: determining that the canister does not have a cannister code and determining that the canister incudes a different cannister code. At least a portion of said modules include one or more of hardware circuits, programmable hardware devices and executable code. The executable code is stored on one or more computer readable storage media.

Another inhaler apparatus for delivering a prescription-controlled, metered-dose, aerosol includes a receiver port of a dispenser. The receiver port is configured to receive a canister with an inhalation substance. The dispenser is configured to dispense the inhalation substance from the canister. The inhaler apparatus includes a wireless data module configured to read encrypted data from and write encrypted data to a memory chip affixed to the canister, an identity input module configured to receive user identity (“ID”) information from a user, and an ID comparison module configured to compare the user ID information with stored ID information. The stored ID information of the user is stored on the dispenser and/or the memory chip of the canister. The inhaler apparatus includes a dispenser module is configured to enable the dispenser to dispense the inhalation substance from the canister in response to the ID comparison module determining that the user ID information matches the stored ID information and configured to lock out the dispenser from dispensing the inhalation substance from the canister in response to the ID comparison module determining that the user ID information does not match the stored ID information. At least a portion of said modules include one or more of hardware circuits, programmable hardware devices and executable code, where the executable code is stored on one or more computer readable storage media.

Another inhaler apparatus for delivering a prescription-controlled, metered-dose, aerosol includes a receiver port of a dispenser. The receiver port is configured to receive a canister with an inhalation substance. The dispenser is configured to dispense the inhalation substance from the canister. The inhaler apparatus includes a wireless data module configured to read encrypted data from and write encrypted data to a memory chip affixed to the canister, a canister code module configured, in response to data read from the memory chip not including a cannister code, to write a cannister code to the memory chip on the canister, where the cannister code is specific to the dispenser, an identity input module configured to receive user identity (“ID”) information from a user, and an ID comparison module configured to compare the user ID information with stored ID information. The stored ID information of the user is stored on the dispenser and/or the memory chip of the canister. The inhaler apparatus includes a dispenser module configured to enable the dispenser to dispense the inhalation substance from the canister in response to determining that the memory chip includes the cannister code and in response to the ID comparison module determining that the user ID information matches the stored ID information and configured to lock out the dispenser from dispensing the inhalation substance from the canister in response to determining that the canister does not have a cannister code, in response to determining that the canister comprises a different cannister code, and in response to the ID comparison module determining that the user ID information does not match the stored ID information. At least a portion of said modules include one or more of hardware circuits, programmable hardware devices and executable code. The executable code is stored on one or more computer readable storage media.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1A is a front view illustrating an inhaler apparatus with a prescription-controlled, metered-dose, aerosol inhaler device, according to various embodiments;

FIG. 1B is a front view illustrating another inhaler apparatus with a prescription-controlled, metered-dose, aerosol inhaler device with an electronic display, according to various embodiments;

FIG. 2A is a front view illustrating the inhaler apparatus of FIG. 1A without a cover, according to various embodiments;

FIG. 2B is a top view illustrating the inhaler apparatus of FIG. 1, according to various embodiments;

FIG. 2C is a bottom view illustrating the inhaler apparatus of FIG. 1, according to various embodiments;

FIG. 2D is a left side view illustrating the inhaler apparatus of FIG. 1, according to various embodiments;

FIG. 2E is a right side view illustrating the inhaler apparatus of FIG. 1, according to various embodiments;

FIG. 3 is a front view of a keyed canister with a memory chip, according to various embodiments;

FIG. 4A is a top view of a keyed receiver top of the keyed canister of FIG. 3, according to various embodiments;

FIG. 4B is a bottom view of a receiver port in the inhaler apparatus of FIG. 1 depicting a keyed shape to receive the keyed receiver top of FIG. 4A, according to various embodiments;

FIG. 5 is a schematic flowchart diagram illustrating a method for using an inhaler apparatus with a prescription-controlled, metered-dose, aerosol inhaler device, according to various embodiments;

FIG. 6 is a chart depicting an example of an oxycodone 15 milligram dose;

FIG. 7 is a chart depicting inhalation doses of oxycodone, according to various embodiments;

FIG. 8A is a front view illustrating yet another inhaler apparatus with a prescription-controlled, metered-dose, aerosol inhaler device locking a canister with a dispenser and a user to the canister, according to various embodiments;

FIG. 8B is a front view illustrating the inhaler apparatus of FIG. 8A with a cover, according to various embodiments;

FIG. 9 is a schematic flowchart diagram illustrating a method for using an inhaler apparatus with a prescription-controlled, metered-dose, aerosol inhaler device and writing a unique canister code to a memory chip of the canister, according to various embodiments;

FIG. 10 is a schematic flowchart diagram illustrating a method for using an inhaler apparatus with a prescription-controlled, metered-dose, aerosol inhaler device in comparing a user ID with a stored user ID and enabling dispensing in case of a match, according to various embodiments; and

FIG. 11 is a schematic flowchart diagram illustrating a method for using an inhaler apparatus with a prescription-controlled, metered-dose, aerosol inhaler device for dispensing a dose of an inhalation substance in response to a correct canister code being read from a memory chip on a canister and in response to a user ID matching a stored user ID, according to various embodiments.

### DETAILED DESCRIPTION

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments.

These features and advantages of the embodiments will become more fully apparent from the following description and appended claims, or may be learned by the practice of embodiments as set forth hereinafter. As will be appreciated by one skilled in the art, aspects of the present invention may be embodied as a system, method, and/or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present invention may take the form of a computer program product embodied in one or more computer readable medium(s) having program code embodied thereon.

Many of the functional units described in this specification have been labeled as modules, in order to more particularly emphasize their implementation independence. For example, a module may be implemented as a hardware circuit comprising custom very large scale integrated (“VLSI”) circuits or gate arrays, off-the-shelf semiconductors such as logic chips, transistors, or other discrete components. A module may also be implemented in programmable hardware devices such as a field programmable gate array (“FPGA”), programmable array logic, programmable logic devices or the like.

Modules may also be implemented in software for execution by various types of processors. An identified module of executable program code (or “executable code” or simply “code”) may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in software, the program code may be stored and/or propagated on in one or more computer readable medium(s).

Furthermore, embodiments may take the form of a program product embodied in one or more computer readable storage devices storing machine readable code, computer readable code, and/or program code, referred hereafter as code. The storage devices, in some embodiments, are tangible, non-transitory, and/or non-transmission.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (“RAM”), a read-only memory (“ROM”), an erasable programmable read-only memory (“EPROM” or Flash memory), a static random access memory (“SRAM”), a portable compact disc read-only memory (“CD-ROM”), a digital versatile disk (“DVD”), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (“ISA”) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (“LAN”) or a wide area network (“WAN”), or the connection may be made to an external computer (for example, through the Internet using an Internet Service Provider). In some embodiments, electronic circuitry including, for example, programmable logic circuitry, field-programmable gate arrays (“FPGA”), or programmable logic arrays (“PLA”) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The schematic flowchart diagrams and/or schematic block diagrams in the Figures illustrate the architecture, functionality, and operation of possible implementations of apparatuses, systems, methods and computer program products according to various embodiments of the present invention. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

The description of elements in each figure may refer to elements of proceeding figures. Like numbers refer to like elements in all figures, including alternate embodiments of like elements.

As used herein, a list with a conjunction of “and/or” includes any single item in the list or a combination of items in the list. For example, a list of A, B and/or C includes only A, only B, only C, a combination of A and B, a combination of B and C, a combination of A and C or a combination of A, B and C. As used herein, a list using the terminology “one or more of” includes any single item in the list or a combination of items in the list. For example, one or more of A, B and C includes only A, only B, only C, a combination of A and B, a combination of B and C, a combination of A and C or a combination of A, B and C. As used herein, a list using the terminology “one of” includes one and only one of any single item in the list. For example, “one of A, B and C” includes only A, only B or only C and excludes combinations of A, B and C.

An inhaler apparatus for delivering a prescription-controlled, metered-dose, aerosol includes a receiver port configured to receive a keyed canister with an inhalation substance. The receiver port includes a key shape to receive a keyed receiver top of the keyed canister and to reject canisters without the keyed receiver top. The inhaler apparatus includes a wireless data module configured to read encrypted data from and write encrypted data to a memory chip affixed to a side of the keyed canister locked into the receiver port, a lockout device configured to prevent, in a locked state, the keyed canister from dispensing a metered dose of the inhalation substance while in a locked position, and a dispenser module configured to maintain the lockout device in the locked state, to read data from the memory chip that includes information including timing for a next metered dose of the inhalation substance, to unlock the lockout device at a time for the next metered dose of the inhalation substance, to lock the lockout device in the locked state in response to a user dispensing the next metered dose, and to signal the wireless data module to write encrypted data to the memory chip comprising a time of dispensing the metered dose. At least a portion of said modules include one or more of hardware circuits, programmable hardware devices and executable code where the executable code is stored on one or more computer readable storage media.

In some embodiments, the keyed receiver top of the keyed canister includes a shape configured to mate with the keyed shape of the receiver port during insertion of the keyed canister and to lock into place when the keyed canister is fully inserted. The keyed shape of the receiver port is configured to reject canisters without a keyed receiver top with the keyed shape. In other embodiments, the keyed shape of the receiver port includes one or more protrusions extending from a center portion of the receiver port. The one or more protrusions are arranged at particular angles around the center portion and the keyed receiver top includes recesses arranged in a same pattern as the one or more protrusions. In other embodiments, the one or more protrusions are configured to rotate into slots of the keyed receiver top to a locked position in response to the keyed canister being rotated with respect to the receiver port.

In some embodiments, the lockout device includes a solenoid configured to extend into a recess of an activation button mechanism where the activation button mechanism is configured to dispense a metered dose of the inhaler substance in response to the user moving the activation button mechanism. In other embodiments, the inhaler apparatus includes a dose timer indicator configured to notify the user in response to reaching a time of the next metered dose of the inhaler substance and/or a dose ready indicator configured to notify the user that the lockout device in an unlocked state. In other embodiments, the dose timer indicator and the dose ready indicator include one or more of a light, a sound, a vibration of the inhaler apparatus, and an electronic display.

In some embodiments, the wireless data module includes an encryption module configured to decrypt encrypted data read from the memory chip and to encrypt data to be written to the memory chip. In other embodiments, the inhaler apparatus includes a lockout failure module configured to send an alert in response to the lockout device failing to lock during the locked state. In other embodiments, the memory chip includes a read/write radio frequency identifier (“RFID”) chip, a memory chip readable and writable using near-field communication (“NFC”); or a memory chip readable and writable using Bluetooth low energy (“BLE”) communications.

In some embodiments, the keyed canister includes a dip tube in fluid communication with a canister valve on the keyed canister and extending to a bottom of an interior of the keyed canister. The dip tube is configured to draw the inhalation substance from the bottom of the interior of the keyed canister during dispensing of a metered dose of the inhalation substance. In other embodiments, the inhaler apparatus includes an encapsulating material encapsulating at least the wireless data module, the lockout device, and the dispenser module. In other embodiments, inserting the keyed canister into the receiver port and rotating the keyed canister with respect to the receiver port to a locked position also positions the memory chip on the keyed canister with a wireless antenna of the wireless data module.

In some embodiments, the inhaler apparatus is configured with a mouthpiece configured to face upward during dispensing of a metered dose, an opening leading to the receiver port positioned so the keyed canister is inserted from a bottom with the keyed receiver top facing upwards and the receiver port positioned downwards, a dispenser button configured to dispense a metered dose from the keyed canister, and an activation button configured to direct the dispenser module to unlock the lockout device. In other embodiments, the inhaler apparatus includes a timeout module configured to lock the lockout device in response to expiration of a timer while the lockout device is unlocked waiting for the user to dispense the next metered dose.

In some embodiments, the dispenser module is configured to, in conjunction with data read from the memory chip, track time to the next metered dose, track a number of doses left to dispense from the keyed canister, control a number of doses to be dispensed by the user at a time of the next dose, and to update data on the memory chip where the updated data includes at least the time of the next dose and/or the number of metered doses left to be dispensed.

Another inhaler apparatus for delivering a prescription-controlled, metered-dose, aerosol includes a receiver port configured to receive a keyed canister with an inhalation substance. The receiver port includes a key shape to receive a keyed receiver top of the keyed canister and to reject canisters without the keyed receiver top. The keyed receiver top of the keyed canister includes a shape configured to mate with the keyed shape of the receiver port during insertion of the keyed canister and to lock into place when the keyed canister is fully inserted. The inhaler apparatus includes a wireless data module configured to read encrypted data from and write encrypted data to a memory chip affixed to a side of the keyed canister locked into the receiver port, an encryption module configured to decrypt encrypted data read from the memory chip and to encrypt data to be written to the memory chip, and a lockout device configured to prevent, in a locked state, the keyed canister from dispensing a metered dose of the inhalation substance while in a locked position. The inhaler apparatus includes a dispenser module configured to maintain the lockout device in the locked state, to read data from the memory chip with information including timing for a next metered dose of the inhalation substance, to unlock the lockout device at a time for the next metered dose of the inhalation substance, to lock the lockout device in the locked state in response to a user dispensing the next metered dose, and to signal the wireless data module to write encrypted data to the memory chip comprising a time of dispensing the metered dose. At least a portion of said modules include one or more of hardware circuits, programmable hardware devices and executable code. The executable code is stored on one or more computer readable storage media.

In some embodiments, the keyed shape of the receiver port includes one or more protrusions extending from a center portion of the receiver port where the one or more protrusions are arranged at particular angles around the center portion, and the keyed receiver top includes recesses arranged in a same pattern as the one or more protrusions. The one or more protrusions are configured to rotate into slots of the keyed receiver top to a locked position in response to the keyed canister being rotated with respect to the receiver port. In other embodiments, the lockout device includes a solenoid configured to extend into a recess of an activation button mechanism. The activation button mechanism is configured to dispense a metered dose of the inhaler substance in response to the user moving the activation button mechanism. In other embodiments, the inhaler apparatus includes a dose timer indicator configured to notify the user in response to reaching a time of the next metered dose of the inhaler substance and/or a dose ready indicator configured to notify the user that the lockout device in an unlocked state. The dose timer indicator and the dose ready indicator include a light and/or an electronic display.

A method for operation of an inhaler apparatus includes receiving, via a receiver port, a keyed canister with an inhalation substance. The receiver port includes a key shape to receive a keyed receiver top of the keyed canister and to reject canisters without the keyed receiver top. The method includes reading, via a wireless data module, encrypted data from and writing encrypted data to a memory chip affixed to a side of the keyed canister locked into the receiver port. The method includes preventing, using a lockout device in a locked state, the keyed canister from dispensing a metered dose of the inhalation substance while in a locked position, maintaining the lockout device in the locked state, reading data from the memory chip that includes information about timing for a next metered dose of the inhalation substance, unlocking the lockout device at a time for the next metered dose of the inhalation substance, and locking the lockout device in the locked state in response to a user dispensing the next metered dose.

Another inhaler apparatus for delivering a prescription-controlled, metered-dose, aerosol includes a receiver port of a dispenser. The receiver port is configured to receive a canister with an inhalation substance. The dispenser is configured to dispense the inhalation substance from the canister. The inhaler apparatus includes a wireless data module configured to read encrypted data from and write encrypted data to a memory chip affixed to the canister and a canister code module configured, in response to data read from the memory chip not including a cannister code, to write a cannister code to the memory chip on the canister. The cannister code is specific to the dispenser. The inhaler apparatus includes a dispenser module configured to enable the dispenser to dispense the inhalation substance from the canister in response to determining that the memory chip includes the cannister code and configured to lock out the dispenser from dispensing the inhalation substance from the canister in response to one of: determining that the canister does not have a cannister code and determining that the canister incudes a different cannister code. At least a portion of said modules include one or more of hardware circuits, programmable hardware devices and executable code. The executable code is stored on one or more computer readable storage media.

In some embodiments, the inhaler apparatus includes an encryption module configured to encrypt the canister code prior to the canister code module writing the canister code to the memory chip of the canister where the encrypted canister code is stored on the memory chip. In other embodiments, the encryption module includes an encryption key specific to dispensers configured to receive the canister. In other embodiments, the encryption key is generated using symmetric encryption and the encryption module includes a common encryption key of asymmetric encryption and the encryption module includes a private encryption key.

In some embodiments, the inhaler apparatus includes an identity input module configured to receive user identity (“ID”) information from a user, and an ID comparison module configured to compare the user ID information with stored ID information. The stored ID information of the user is stored on the dispenser and/or the memory chip of the canister. In the embodiments, the dispenser module is configured to enable the dispenser to dispense the inhalation substance from the canister in response to determining that the memory chip includes the cannister code and in response to the ID comparison module determining that the user ID information matches the stored ID information and configured to lock out the dispenser from dispensing the inhalation substance from the canister in response to one or more of: determining that the canister does not have a cannister code, determining that the canister comprises a different cannister code, and the ID comparison module determining that the user ID information does not match the stored ID information.

In other embodiments, the identity input module receives the ID information from the user via: a keypad on the dispenser, where the user ID information includes a code input by the user via the keypad, a fingerprint reader on the dispenser, where the user ID information includes a fingerprint of the user, a camera on the dispenser, where the user ID information includes an image of the user, and/or a retina scanner on the dispenser, where the user ID information includes a retina scan of the user. In other embodiments, a dispensing authority stores the stored ID information of the user on the memory chip when the canister is dispensed to the user and/or on the dispenser when the dispenser is provided to the user.

In other embodiments, the inhaler apparatus includes a dose dispenser module configured to signal an internal actuator to dispense a dose of the inhalation substance from the canister in response to the dispenser module enabling the dispenser to dispense the inhalation substance. The dose dispenser module requires no further action from the user. In other embodiments, the dose dispenser module dispenses the dose after a time delay. In some embodiments, the memory chip includes a read/write radio frequency identifier (“RFID”) chip, a memory chip readable and writable using near-field communication (“NFC”), or a memory chip readable and writable using Bluetooth low energy (“BLE”) communications.

An inhaler apparatus for delivering a prescription-controlled, metered-dose, aerosol includes a receiver port of a dispenser. The receiver port is configured to receive a canister with an inhalation substance. The dispenser is configured to dispense the inhalation substance from the canister. The inhaler apparatus includes a wireless data module configured to read encrypted data from and write encrypted data to a memory chip affixed to the canister, an identity input module configured to receive user identity (“ID”) information from a user, and an ID comparison module configured to compare the user ID information with stored ID information. The stored ID information of the user is stored on the dispenser and/or the memory chip of the canister. The inhaler apparatus includes a dispenser module is configured to enable the dispenser to dispense the inhalation substance from the canister in response to the ID comparison module determining that the user ID information matches the stored ID information and configured to lock out the dispenser from dispensing the inhalation substance from the canister in response to the ID comparison module determining that the user ID information does not match the stored ID information. At least a portion of said modules include one or more of hardware circuits, programmable hardware devices and executable code, where the executable code is stored on one or more computer readable storage media.

In some embodiments, the inhaler apparatus includes a canister code module configured, in response to data read from the memory chip not including a cannister code, to write a cannister code to the memory chip on the canister. The cannister code is specific to the dispenser. The dispenser module is configured to enable the dispenser to dispense the inhalation substance from the canister in response to determining that the memory chip includes the cannister code and in response to the ID comparison module determining that the user ID information matches the stored ID information and configured to lock out the dispenser from dispensing the inhalation substance from the canister in response to one or more of determining that the canister does not have a cannister code, determining that the canister includes a different cannister code, and the ID comparison module determining that the user ID information does not match the stored ID information.

In other embodiments, the inhaler includes an encryption module configured to encrypt the canister code prior to the canister code module writing the canister code to the memory chip of the canister. The encrypted canister code is stored on the memory chip. In other embodiments, the encryption module includes an encryption key specific to dispensers configured to receive the canister.

In some embodiments, the identity input module receives the ID information from the user via: a keypad on the dispenser, where the user ID information includes a code input by the user via the keypad, a fingerprint reader on the dispenser, where the user ID information includes a fingerprint of the user, a camera on the dispenser, where the user ID information includes an image of the user, and/or a retina scanner on the dispenser, where the user ID information includes a retina scan of the user. In other embodiments, the stored ID information is stored on the memory chip of the canister and a dispensing authority stores the stored ID information of the user on the memory chip when the canister is dispensed to the user.

In some embodiments, the inhaler apparatus includes a dose dispenser module configured to signal an internal actuator to dispense a dose of the inhalation substance from the canister in response to the dispenser module enabling the dispenser to dispense the inhalation substance. The dose dispenser module requires no further action from the user. In other embodiments, the dose dispenser module dispenses the dose after a time delay.

An inhaler apparatus for delivering a prescription-controlled, metered-dose, aerosol includes a receiver port of a dispenser. The receiver port is configured to receive a canister with an inhalation substance. The dispenser is configured to dispense the inhalation substance from the canister. The inhaler apparatus includes a wireless data module configured to read encrypted data from and write encrypted data to a memory chip affixed to the canister, a canister code module configured, in response to data read from the memory chip not including a cannister code, to write a cannister code to the memory chip on the canister, where the cannister code is specific to the dispenser, an identity input module configured to receive user identity (“ID”) information from a user, and an ID comparison module configured to compare the user ID information with stored ID information. The stored ID information of the user is stored on the dispenser and/or the memory chip of the canister. The inhaler apparatus includes a dispenser module configured to enable the dispenser to dispense the inhalation substance from the canister in response to determining that the memory chip includes the cannister code and in response to the ID comparison module determining that the user ID information matches the stored ID information and configured to lock out the dispenser from dispensing the inhalation substance from the canister in response to determining that the canister does not have a cannister code, in response to determining that the canister comprises a different cannister code, and in response to the ID comparison module determining that the user ID information does not match the stored ID information. At least a portion of said modules include one or more of hardware circuits, programmable hardware devices and executable code. The executable code is stored on one or more computer readable storage media.

In some embodiments, the identity input module receives the ID information from the user via: a keypad on the dispenser, where the user ID information includes a code input by the user via the keypad, a fingerprint reader on the dispenser, where the user ID information includes a fingerprint of the user, a camera on the dispenser, where the user ID information includes an image of the user, and/or a retina scanner on the dispenser, where the user ID information includes a retina scan of the user.

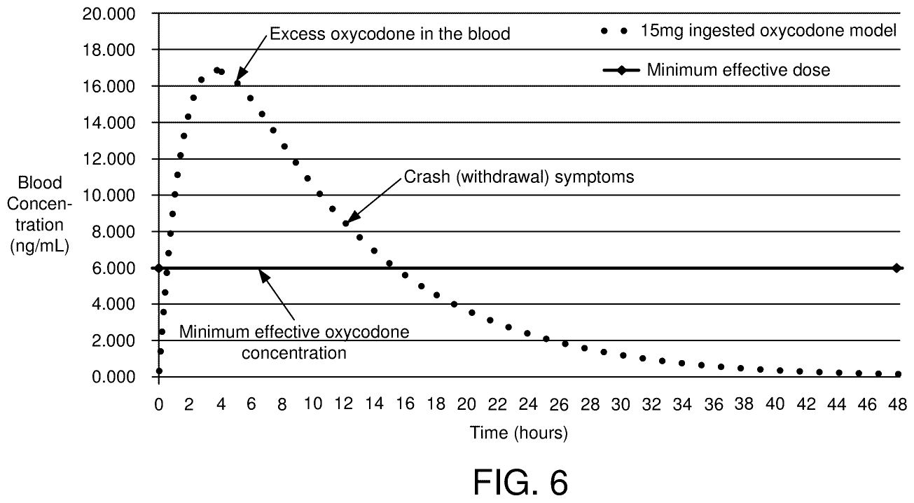

Opioids are effective at treating moderate to severe pain. However, opioids are highly addictive. Time released formulations have been developed, but take 40-60 minutes to take effect. A better method for delivering opioids, medications, and other substances that is safe, fast, and effective is desirable. One problem with use of opioids in pill form or a liquid form is the amount of opioid in the body of a person taking the pill or liquid just after ingestion. One type of opioid is oxycodone. FIG. 6 is a chart depicting an example of an oxycodone 15 milligram (“mg”) dose. The minimum effective dose is a blood concentration of about 6 nanograms (“ng”) per milliliter (“mL”), which is depicted as a solid line. After about 4 hours, the blood concentration of oxycodone in the person's blood is about 17 ng/mL (the dashed line), which is very much in excess of the minimum effective dose of oxycodone. This peak at about 4 hours is followed by a rapid decrease over the next few hours, which can result in withdrawal symptoms.

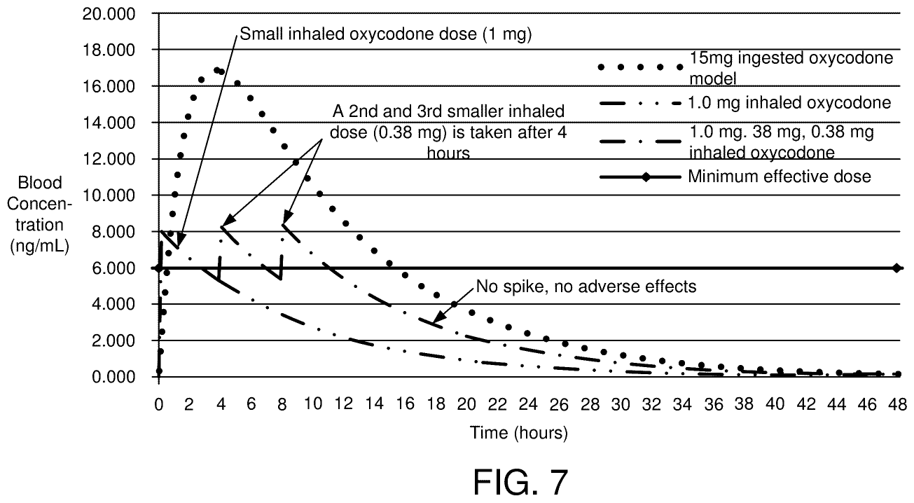

FIG. 7 is a chart depicting inhalation doses of the oxycodone, according to various embodiments. The 15 mg dose of FIG. 6 is also depicted for reference. An initial inhalation dose is 1.0 mg of oxycodone followed by two inhalation doses of 0.38 mg every 4 hours. Note that the initial and subsequent inhalation doses only reach a maximum blood concentration of about 8 ng/mL, but also reach the 8 ng/mL quickly. The lower maximum of each dose helps to prevent the withdrawal symptoms that occur with a pill or liquid and administering each dose by an inhaler speeds up the oxycodone in the bloodstream of the person.

However, traditional inhalers are prone to being misused where a person could take multiple metered doses at a time, could take metered doses at a rate faster than prescribed, etc. What is needed is an inhaler apparatus that electronically controls when metered doses are administered and has mechanisms to prevent a user from tampering with the inhaler apparatus. The embodiments described below describe a prescription-controlled, metered-dose, aerosol inhaler apparatus. A purpose of this invention is to describe technologies that will make the prescription-controlled devices safe, tamper resistant, effective for multiple drugs and Federal Drug Administration (“FDA”) 510k compliant.

A purpose of the present invention is to improve the safety and performance of a metered dose aerosol inhaler device and reduce the likelihood of abuse of the inhalation formula containing at least one active ingredient that may or may not be chemically or psychologically addictive. After inhaling the prescribed metered dose, the inhaler device uses a lock-out mechanism, where the device cannot be used again during a factory programed, prescribed interval. The inhalation device also uses keyed, tamper resistant cartridges for the formula containing the active ingredient, where the formula cannot be dispensed outside of the inhalation device.

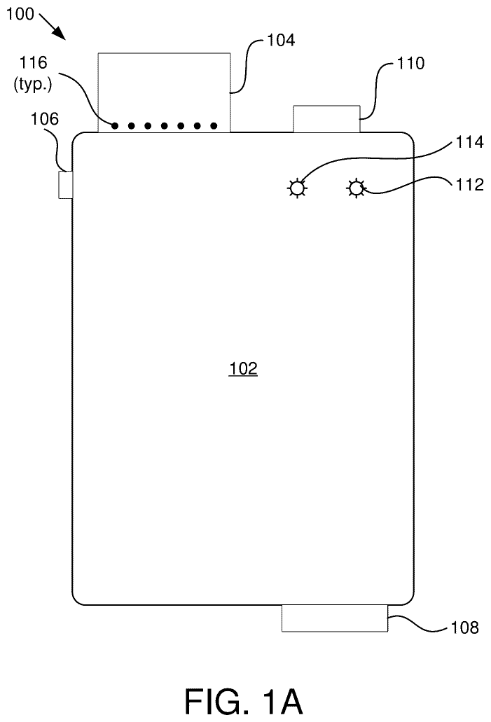

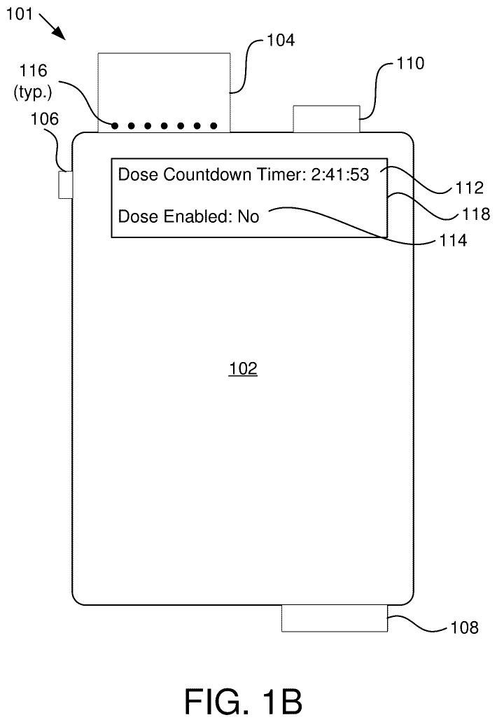

FIG. 1A is a front view illustrating an inhaler apparatus 100 with a prescription-controlled, metered-dose, aerosol inhaler device, according to various embodiments. In some embodiments, the inhaler apparatus 100 is configured to be handheld. The inhaler apparatus 100 includes a propellant control unit 102 that includes a receiver port configured to receive a keyed canister 108 that includes an inhalation substance. The receiver port includes a key shape to receive a keyed receiver top of the keyed canister 108 and to reject canisters without the keyed receiver top. The propellant control unit 102 also includes a wireless data module configured to read encrypted data from and write encrypted data to a memory chip affixed to a side of the keyed canister 108 locked into the receiver port.

The propellant control unit 102 also includes a lockout device configured to prevent, in a locked state, the keyed canister 108 from dispensing a metered dose of the inhalation substance while in a locked position. In some embodiments the lockout device includes a solenoid that extends into a recess of an activation button mechanism configured to prevent an activation button 110 from being pressed to dispense a metered dose of the inhalation substance, and retracts to allow the activation button 110 to be pressed by a user to dispense a metered dose of the inhalation substance. The activation button mechanism includes a rod or other structure below the activation button 110 that extends to a canister valve of the keyed canister 108.

The propellant control unit 102 also includes a dispenser module configured to maintain the lockout device in the locked state, to read data from the memory chip that includes information about timing for a next metered dose of the inhalation substance, to unlock the lockout device at a time for a next metered dose of the inhalation substance, and to lock the lockout device in the locked state in response to a user dispensing the next metered dose.

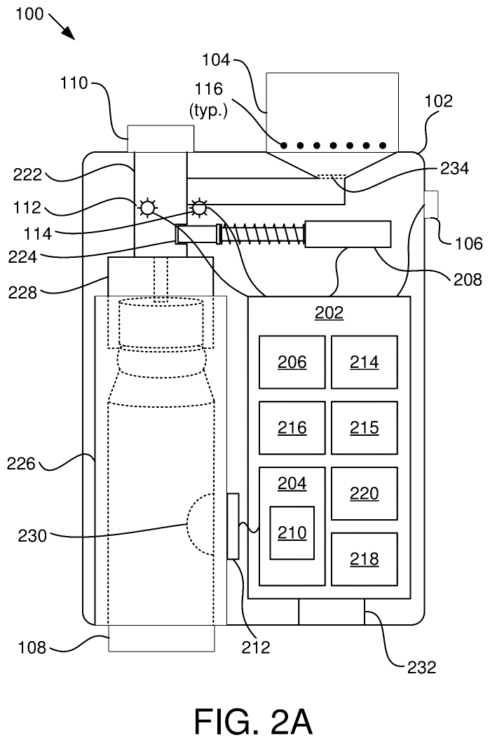

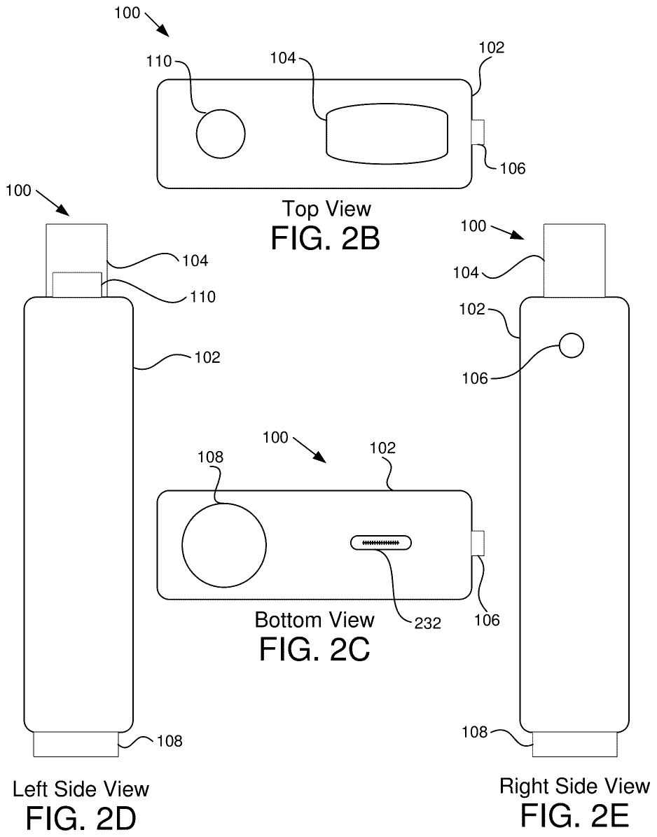

The inhaler apparatus 100 includes a mouthpiece 104 configured to allow a user to hold the mouthpiece 104 in their mouth while pressing the activation button 110 to dispense a metered dose. In some embodiments, the mouthpiece 104 is perforated with small holes 116 to allow some air to flow into the inner portion of the mouthpiece 104 along with the inhalation substance during dispensing of a metered dose. In some embodiments, the mouthpiece 103 has a shape to more easily fit a user's mouth, as depicted in FIG. 2B. In some embodiments, the mouthpiece 104 includes a nozzle at the bottom of the mouthpiece 104 where the nozzle aerosolizes the inhalation substance.

The inhaler apparatus 100 includes, in some embodiments, a trigger button 106 configured to trigger an inhalation timing cycle after a lockout period has expired. The lockout period is a time between metered doses while the lockout device prevents the activation button 110 from dispensing a metered dose. In some embodiments, the trigger button 106 is a mechanical button that closes a contact. In other embodiments, the trigger button is an electronic switch that senses a user touch or press and uses a transistor or other circuitry to send a signal. In other embodiments, the trigger button 106 includes a fingerprint reader that verifies the identity of a user before sending a signal. While the trigger button 106 is depicted on the right side of the propellant control unit 102, in other embodiments the trigger button 106 is located elsewhere on the propellant control unit 102.

The inhaler apparatus 100 includes a keyed canister 108 that includes a keyed receiver top configured to match a key shape of the receiver port of the propellant control unit 102. The keyed canister 108 includes an inhalation substance used in a metered dose to the user. In some embodiments, the inhalation substance is an aerosol. In some embodiments, the inhalation substance includes a medication to be dispensed by inhalation of the user through the mouthpiece 104. In other embodiments, the inhalation substance includes one or more prescribed or other-the-counter active ingredients. In other embodiments, the inhalation substance includes a supplement to be dispensed in a metered dose. In various embodiments, the inhalation substance includes alcohol, a propellant, an inert gas, a preservative, or other substance typically found in an inhalation substance.

In some embodiments, the inhalation substance is water based and the mouthpiece 104 includes a nebulizer configured to nebulize the inhalation substance. In some embodiments, the nebulizer creates droplets of the inhalation substance that are in the range of 0.1 to 100 micrometers (“μm”). In other embodiments, the nebulizer creates droplets of the inhalation substance that are in the range of 1 to 10 μm. In some embodiments, the nebulizer is an ultrasonic nebulizer, which may come in the form of a piezoelectric nebulizer. A piezoelectric nebulizer vibrates a small plate to nebulize the inhalation substance. In other embodiments, the nebulizer is a jet nebulizer. In other embodiments, the nebulizer is a mesh nebulizer that forces the inhalation substance through a fine mesh to form the inhalation substance into an aerosol. One of skill in the art will recognize other forms of the nebulizer when the inhalation substance is water based or is otherwise formulated to be used with a nebulizer.

The keyed canister 108 includes the keyed receiver top, which is unique to the receiver port of the propellant control unit 102 to prevent the keyed canister 108 from being used in inhaler devices that don't have the key shape in the receiver port of the inhaler. In some embodiments, the keyed canister 108 and propellant control unit 102 are unique to a particular company or manufacturer and the key shape of the receiver port may be used for any keyed canister 108 with a keyed receiver top that matches the key shape of the receiver port. In other embodiments, the keyed receiver top of the keyed canister 108 is unique for a particular inhalation substance and a matching propellant control unit 102 is configured with a receiver port with a key shape that matches the keyed canister 108 of the particular inhalation substance in the keyed canister 108. In other embodiments, the keyed receiver top of keyed canisters 108 are specific to a particular user that has a matching propellant control unit 102 with a receiver port with a key shape that matches the keyed receiver tops of the keyed canister 108 of the user.

The inhaler apparatus 100 includes an activation button 110 configured to dispense a metered dose from the keyed canister 108 in response to the lockout device being out of the locked state so that the activation button 110 is able to depressed by the user to press on a cartridge valve of the keyed canister 108, which releases a metered dose of the inhalation substance. In some embodiments, the activation button 110 is aligned with the cartridge valve of the keyed canister 108 with a rod extending from the activation button 110 to the cartridge valve. In other embodiments, the activation button 110 is offset from the cartridge valve and a rod to from the activation button 110 to the canister valve includes an offset. In other embodiments, the activation button 110 is on a side of the propellant control unit 102 and two or more rods along with linkage allow pressing the activation button 110 to depress the canister valve.

In some embodiments, the inhaler apparatus 100 includes a dose timer indicator 112 configured as a light. In some embodiments, the light is a light emitting diode (“LED”), a fluorescent lamp, an incandescent lamp, or other type of lamp capable of emitting light. In some embodiments, the dose timer indicator 112 in the form of a light is configured to remain off until a time for a next metered dose of the inhalation substance and is configured to light up when a time for a next metered dose has arrived. In some embodiments, the inhaler apparatus 100 includes a clock and a dose time comparator that determines if the time for the next metered dose has arrived. In other embodiments, the inhaler apparatus 100 uses clock cycles, a countdown timer, or another metric to determine if a time for a next metered dose has arrived or not.

In some embodiments, after reaching a time for a next dose, the inhaler apparatus 100 plays a sound and/or vibrates to signal to the user that a time has been reached for a next metered dose. In such embodiments, the inhaler apparatus 100 includes a speaker and/or a vibration device. In some embodiments, the dose timer indicator 112 light or signals at the same time as playing a sound and/or vibrating the inhaler apparatus 100.

In some embodiments, the inhaler apparatus 100 includes a dose ready indicator 114 configured to notify the user that the lockout device in an unlocked state. Once the dose timer indicator 112 has indicated that the next metered dose is available, the user presses the trigger button 106, which is configured to place the lockout device in an unlocked state and to activate the dose ready indicator 114, which notifies the user that the user can press the activation button 110 to administer the next metered dose. The inhaler apparatus 100 of FIG. 1A depicts the dose ready indicator 114 as a light, which may be an LED or other device that emits light. In some embodiments, pressing the trigger button 106 also triggers playing a sound and/or vibrating the inhaler apparatus 100.

FIG. 1B is a front view illustrating another inhaler apparatus 101 with a prescription-controlled, metered-dose, aerosol inhaler device with an electronic display, according to various embodiments. The inhaler apparatus 101 is substantially similar to the inhaler apparatus 100 of FIG. 1A except that the light of the dose timer indicator 112 and the light of the dose ready indicator 114 are replaced with an electronic display 118. In the embodiments of the inhaler apparatus 101 of FIG. 1B, the dose timer indicator 112 is a message on the electronic display 118 indicating how much time to a next metered dose. Likewise, in the embodiments of the inhaler apparatus 101 of FIG. 1B, the dose ready indicator 114 is a message on the electronic display 118. In other embodiments, the dose timer indicator 112 is a different message while displaying when the dose countdown timer has expired. In FIG. 1B, the dose ready indicator 114 is a message of “Dose Enabled: No,” which would change to “Dose Enabled: Yes” after the user presses the trigger button 106 while the dose timer indicator 112 is active or lit. In other embodiments, the message is different indicating that the user is able to dispense a next metered dose.

FIG. 2A is a front view illustrating the inhaler apparatus 100 of FIG. 1A without a cover, FIG. 2B is a top view illustrating the inhaler apparatus 100 of FIG. 1, FIG. 2C is a bottom view illustrating the inhaler apparatus 100 of FIG. 1, FIG. 2D is a left side view illustrating the inhaler apparatus 100 of FIG. 1, and FIG. 2E is a right side view illustrating the inhaler apparatus 100 of FIG. 1, according to various embodiments. FIGS. 2A-2E depict the propellant control unit 102, the mouthpiece 104, the trigger button 106, the keyed canister 108, and the activation button 110, are substantially similar to those of inhaler apparatuses 100, 101 of FIGS. 1A and 1B. As used herein, an inhaler apparatus 100 also includes the inhaler apparatus 101 of FIG. 1B. The dose timer indicator 112 and the dose ready indicator 114 are substantially similar to those of the inhaler apparatus 100 of FIG. 1A.

FIG. 2A depicts a microcontroller 202 that controls the inhaler apparatuses 100, 101 of FIGS. 1A and 1B. FIG. 2B In various embodiments, the microcontroller 202 includes one or more processors, a programmable hardware device, hardware circuits, a printed circuit board (“PCB”), and/or the like. The microcontroller 202 also depicts a wireless data module 204 with an encryption module 210, a connected wireless antenna 212, a lockout device module 206, a lockout device 208, a dispenser module 214, a lockout failure module 216, a rechargeable battery 218, and a voltage converter 220, a rod 222, a notch 224, a canister holder 226, a receiver port 228, a memory chip 230, a charging port 232, and a nozzle 234, which are described below.

In some embodiments, the microcontroller 202 includes one or more processors configured to execute code stored on computer readable storage media, such as non-volatile memory placed on a PCB of the microcontroller 202 or elsewhere in the inhaler apparatus 100. The PCB of the microcontroller 202, in other embodiments, includes volatile memory in communication with the one or more processors. Note that the wireless data module 204, the encryption module 210, the lockout device module 206, the dispenser module 214, and/or the lockout failure module 216 are stored in computer readable storage media and are executed by the processor. In other embodiments, all or a portion of the wireless data module 204, the encryption module 210, the lockout device module 206, the dispenser module 214, and/or the lockout failure module 216 include hardware circuits. In other embodiments, the microcontroller 202 is implemented using a programmable hardware device, such as an FPGA or programmable array logic, and all or a portion of the wireless data module 204, the encryption module 210, the lockout device module 206, the dispenser module 214, and/or the lockout failure module 216 are implemented on the programmable hardware device.

The wireless data module 204 is configured to read encrypted data from and write encrypted data to a memory chip 230 affixed to a side of the keyed canister 108 locked into the receiver port 228. In various embodiments, the memory chip 230 includes the number of pulses per metered dose, the number of remaining metered doses, the prescribed time between metered doses, the data and time of the last administered metered dose, the expiration date, and/or other pertinent data.

In some embodiments, the memory chip 230 is a read/write radio frequency identifier (“RFID”) chip, the wireless antenna 212 is an RFID reader/writer, and the wireless data module 204 communicates accordingly. In other embodiments, the memory chip 230 and the wireless antenna 212 communicate using near-field communication (“NFC”) and the wireless data module 204 communicates with the memory chip 230 using NFC. In other embodiments, the memory chip 230, the wireless antenna 212, and the wireless data module 204 use Bluetooth low energy (“BLE”) to communicate. In other embodiments, the memory chip 230, wireless antenna 212, and the wireless data module 204 use another current or future communications protocol. While the memory chip 230 is depicted as adjacent to the wireless antenna 212, in other embodiments, the memory chip 230 is rotated to another position and the wireless antenna 212 is able to read from and write to the memory chip 230.

In some embodiments, the wireless data module 204 includes an encryption module 210 configured to decrypt encrypted data read from the memory chip 230 and to encrypt data to be written to the memory chip 230. In some embodiments, the encryption module 210 is programmed with a password, also referred to herein as an encryption key, to encrypt data from the microcontroller 202 before the wireless data module 204 writes the encrypted data to the memory chip 230. In other embodiments, the encryption module 210 receives encrypted data from the wireless data module 204 that has been read from the memory chip 230 and decrypts the encrypted data using the encryption key. The decrypted data may then be used by the microcontroller 202. In other embodiments, the encryption module 210 is also used to encrypt and decrypt communications between the inhaler apparatus 100 and an outside party, such as the manufacturer of the inhaler apparatus 100, a system administrator, etc.

In some embodiments, the encryption module 210 uses symmetric encryption along with a single encryption key to encrypt and decrypt data. The single encryption key, in some embodiments, is distributed in various inhaler apparatuses 100 so that a keyed canister 108 with a memory chip 230 with encrypted data using the encryption key can be removed from an inhaler apparatus 100 and inserted into another inhaler apparatus 100, which is able to decrypt the encrypted data in the memory chip 230. In other embodiments, the encryption module 210 uses asymmetric encryption with a public encryption key used to encrypt data and a separate private encryption key used to decrypt data. In the embodiments, the public key for encryption may be distributed freely while the private key is programmed into the inhaler apparatus 100 at the factory or other secure environment.

In other embodiments, the encryption module 210 uses public key infrastructure (“PKI”) as the encryption protocol. PKI uses digital certificates and asymmetric key pairs to authenticate uses and devices within a network. Other encryption protocols include transport layer security (“TLS”)/secure socket layer (“SSL”) which ensures communications between a client and a server are kept secure, such as communications between the inhaler apparatus 100 and a manufacturer. Another encryption protocol which may be used by the encryption module 210 is internet protocol security (“IPsec”). One of skill in the art will recognize other appropriate encryption protocols for the encryption module 210.

The microcontroller 202 is connected to a wireless antenna 212, which is used by the wireless data module 204 to wirelessly transmit and receive data from the memory chip 230 on the keyed canister 108. In some embodiments, the wireless antenna 212 is configured for near field communications (“NFC”). In other embodiments, the wireless antenna 212 is configured for Bluetooth or BLE communications. In other embodiments, the wireless antenna 212 is configured for another wireless technology suitable for very close communications.

In some embodiments, the memory chip 230 is positioned to be next to the wireless antenna 212 after the keyed canister 108 is locked into place. In some embodiments, a keyed shape of the receiver port 228 and the keyed receiver top are configured so that as the keyed canister 108 is inserted and then locked, for example by twisting the keyed canister 108, so the memory chip 230 is positioned adjacent to the wireless antenna 212. In other embodiments, the memory chip 230 not adjacent to the wireless antenna 212 and the wireless antenna 212 is able to read data from and write da ta to the memory chip 230. In some embodiments, the keyed shape of the receiver port 228 includes one or more protrusions extending from a center portion of the receiver port 228. The one or more protrusions are arranged at particular angles around the center portion and the keyed receiver top includes recesses arranged in a same pattern as the one or more protrusions. In other embodiments, the one or more protrusions are configured to rotate into slots of the keyed receiver top to a locked position in response to the keyed canister 108 being rotated with respect to the receiver port 228. In other embodiments, the keyed receiver top includes other shapes capable of functioning as a key or able to receive a key on the receiver port 228.

The microcontroller 202 includes, in some embodiments, a lockout device module 206 configured to control a lockout device 208 to enable a locked state and an unlocked state. The locked state prevents the activation button 110 from being used to dispense a metered dose. In the unlocked state, the activation button 110 is able to be pressed to dispense a metered dose from the keyed canister 108. In some embodiments, the lockout device module 206 is configured to place the lockout device 208 in the unlocked state at the start of a next metered dose time. In some embodiments, the start of the next metered dose time is after reaching a time for a next metered dose. In some embodiments, the lockout device module 206 includes a power transistor to energize and to de-energize the lockout device 208. Where the lockout device 208 is a solenoid, the power transistor is configured to turn on and turn off the solenoid when directed by the dispenser module 214.

In some embodiments, the activation button 110 is electronic and provides a signal when pressed and the rod 222 is replaced by a solenoid in contact with the canister valve of the keyed canister 108. In the embodiments, the lockout device 208 may be a switch or other mechanism that is able to prevent a signal from the activation button 110 from reaching the solenoid connected to the canister valve.

In some embodiments, the wireless data module 204 reads the memory chip 230 to determine a time for the next metered dose and the dispenser module 214 is configured to determine when the time for the next metered dose is reached. When the time for the next metered dose is reached, the dispenser module 214, in some embodiments, notifies the lockout device module 206, which sends a signal to the lockout device 208 to unlock the activation button 110. In other embodiments, when the time for the next metered dose is reached, the dispenser module 214 activates the dose timer indicator 112 and then when the trigger button 106 is depressed, the dispenser module 214 notifies the lockout device module 206, which sends a signal to the lockout device 208 to unlock the activation button 110.

In some embodiments, the lockout device module 206 is configured to transmit a signal to the lockout device 208 to enter the locked state. In some embodiments, the lockout device module 206 transmits the signal to enter the locked state in response to a metered dose being administered in the case of a single metered dose or to enter the locked state in response to a final metered dose of a multidose sequence. In other embodiments, the lockout device module 206 is configured to transmit a signal to the lockout device 208 to enter the locked state after expiration of a lockout timer. In some embodiments, the lockout timer is started in response to the trigger button 106 being activated to prevent the trigger button 106 from activating the unlocked state for a prolonged period of time. In some embodiments, the lockout device module 206 also deactivates the dose ready indicator 114 at the end of the lockout timer. In some embodiments where the lockout device 208 is a solenoid, the low power or no power state of the solenoid includes having the tip of the solenoid in the notch 224 in the locked state and applying a voltage to the solenoid pulls the tip of the solenoid out of the notch 224 in the unlocked state, or vice versa.

The microcontroller 202 includes dispenser module 214 configured, in conjunction with the lockout device module 206, to maintain the lockout device 208 in the locked state, to read data from the memory chip 230 that includes information including timing for a next metered dose of the inhalation substance, to unlock the lockout device 208 at a time for the next metered dose of the inhalation substance, and to lock the lockout device 208 in the locked state in response to a user dispensing the next metered dose. Where the memory chip 230 of the keyed canister 108 includes information that indicates more than one metered dose is to be given at a time, in some embodiments, after a metered dose the dispenser module 214 unlocks the lockout device 208 for each subsequent dose in the multidose sequence.

In some embodiments, the dispenser module 214 is configured to communicate with the wireless data module 204, the lockout device module 206, and other functions of the microcontroller 202. In some embodiments, the dispenser module 214 decrements the remaining doses counter in response to a metered dose being dispensed. In some embodiments, the dispenser module 214 is configured to signal the wireless data module 204 to write encrypted data to the memory chip 230 that includes a time of dispensing the metered dose. The dispenser module 214 may also signal the wireless data module 204 to write other pertinent data after a metered dose, such as a new count of remaining doses.

In some embodiments, the dispenser module 214 includes a timing circuit. In some embodiments, the timing circuit is factory programmable. In some embodiments, the timing circuit counts from a previous dose to reach a time between metered doses. In some embodiments, the time between metered doses is encrypted information read from the memory chip 230 and decrypted. In some embodiments, the timing circuit includes a real-time clock and the timing circuit calculates a next metered dose based on a time of a previous metered dose and a time between metered doses. In the embodiments, the timing circuit checks a current time and date to determine if the current date and time is greater than the date and time for the next metered dose. In some embodiments, the timing circuit checks every minute to determine if it is time for a next metered dose. In other embodiments, the timing circuit uses a different interval for checking to determine if it is time for the next metered dose. One of skill in the art will recognize other forms of the timing circuit.

In some embodiments, at a time of a next metered dose, in some cases each metered dose is part of a multidose prescription. The multidose prescription may include two doses, three doses, or more. In some embodiment, after a first metered dose the lockout device 208 is set to a locked state preventing the user from dispensing another dose and the dose ready indicator 114 turns off. Where there is a second dose in a multidose prescription, after a prescribed amount of time the dose ready indicator 114 turns back on and the lockout device module 206 puts the lockout device 208 in the unlocked state allowing the user to dispense a second metered dose. After the second metered dose, the dose ready indicator 114 goes off again and stays off if there are no more prescribed metered doses in the multidose prescription.

If there are one or more metered doses in the multidose prescription, after the prescribed amount of time the dose ready indicator 114 turns back on and the lockout device module 206 sends a signal to the lockout device 208 to go to the unlocked state ready for the user to dispense another metered dose. At the end of the inhalation cycle of a multidose prescription, the dispenser module 214 decrements the number of remaining metered doses, turns off the dose timer indicator 112, and determines a time for a next metered dose. The dispenser module 214 commands the wireless data module 204 to encrypt the time for the next metered dose, the time of the current dose, the number of remaining doses, and/or any other relevant information, and writes the encrypted data to the memory chip 230.

Where there is no keyed canister 108 in the propellant control unit 102, the inhaler apparatus 100 will not operate. The memory chip 230 on a keyed canister 108 with its encrypted and encryption key protected data enables an inhaler apparatus 100 to be used with keyed canisters 108 having formulas containing different active ingredients, while still preventing misuse or abuse. In other words, if the inserted keyed canister 108 is changed, the new keyed canister 108 cannot be used until the prescribed time after the last use of the keyed canister 108.

In some embodiments, the dispenser module 214 transmits an alert, displays an alert, turns a light a particular color, causes a light to blink, etc. when the remaining metered doses is less than a particular value, such as 10 metered doses. The light may be the dose timer indicator 112 and/or the dose ready indicator 114. In other embodiments, the dispenser module 214 lists a message on an electronic display 118 indicating that the amount of remaining metered doses is low, and in some embodiments, provides instructions on how to reorder a keyed canister 108.

In some embodiments, the dispenser module 214 sends a signal to cause the lockout device 208 to go to the locked state in response to the number of remaining metered doses dropping to zero. In other embodiments, the dispenser module 214 sends a signal to cause the lockout device 208 to go to the locked state in response to the keyed canister 108 reaching an expiration date. In other embodiments, when the number of metered doses is zero or the expiration date is arrived at, the dispenser module 214 send a message to the electronic display 118, lights a light a particular color, causes a light to blink in a particular pattern, etc.

In some embodiments, the microcontroller 202 includes a timeout module 215 configured to lock the lockout device 208 in response to expiration of a timer while the lockout device 208 is unlocked waiting for the user to dispense the next metered dose. The timeout module 215 is configured to not allow the lockout device 208 to stay in a locked state indefinitely where the user has not taken the next metered dose.

In some embodiments, the microcontroller 202 includes a lockout failure module 216 configured to send an alert in response to the lockout device 208 failing to lock during the locked state. In some embodiments, the lockout device 208 is configured to send a signal when in the locked state and the lockout failure module 216 reads the signal from the lockout device 208 and determines that the lockout device 208 should be in the locked state and then sends the alert. In some embodiments, the lockout failure module 216 turns the dose timer indicator 112 and/or the dose ready indicator 114 red or another color reserved for an error condition. In other embodiments, the activates another error indicator, such as a separate light or a message on an electronic display 118 indicating the error condition.

In some embodiments, the lockout device 208 is a solenoid as depicted in FIG. 2A. In the embodiments, a lockout pin of the solenoid is configured to extend into a notch 224 or other recess, hole, etc. in a rod 222 that extends from the activation button 110 to the receiver port 228 and to the cartridge valve of the keyed canister 108. While the lockout pin of the solenoid is extended into the notch 224, the rod 222 is locked so that the activation button 110 is in the locked state and a user is unable to dispense a metered dose of the inhalation substance. When the lockout pin of the solenoid is withdrawn from the notch 224, the lockout device 208 is in the unlocked state and the user is able to press on the activation button 110 and the rod 222 moves and is able to press on the cartridge valve of the keyed canister 108 to dispense a metered dose of the inhalation substance.

In other embodiments, the lockout device 208 is in another form, such as a device that rotates where an end of the lockout device 208 rotates out of a recess 224 in the rod 222 into an unlocked state and rotates back into the recess 224 and into a locked state. One of skill in the art will recognize other forms of a lockout device 208 that enables the locked state and enables the unlocked state.

The inhaler apparatus 100 includes, in some embodiments, a rechargeable battery 218 and/or a voltage converter 220, which are used to power the microcontroller 202, the lockout device 208, the wireless antenna 212, the dose timer indicator 112, the dose ready indicator 114, the trigger button 106, and other electronic components of the inhaler apparatus 100. In some embodiments, the rechargeable battery 218 is located on a PCB of the microcontroller 202. In other embodiments, the rechargeable battery 218 is located elsewhere in the inhaler apparatus 100.

In some embodiments, the voltage converter 220 is configured to convert a voltage from the rechargeable battery 218 to another voltage. In some embodiments, the rechargeable battery 218 is at 3.7 volts (“V”), which is an input to the voltage converter 220, which converts the voltage to 5 V. Often 5 V is used for larger components, such as the lockout device 208 or other devices such as the microcontroller 202. In some embodiments, some electronic components of the inhaler apparatus 100 use 3.7 V from the battery. In other embodiments, the voltage converter 220 outputs more than one voltage. In some embodiments, a second voltage available from the voltage converter 220 is lower than the battery voltage. In some embodiments, the voltage converter 220 includes surge protection to prevent damage from over voltage, includes overcurrent protection, short circuit protection, low voltage protection, and the like.

The inhaler apparatus 100 includes a canister holder 226 configured for the keyed canister 108 to fit into. At a top of the canister holder 226 is the receiver port 228. The canister holder 226, in some embodiments, is configured with a particular diameter just larger than an outer diameter of the keyed canister 108. In some embodiments, the canister holder 226 is sized so that a portion of the keyed canister 108 protrudes out of the bottom of the propellant control unit 102 to allow a user to rotate the keyed canister 108 and to pull the keyed canister 108 from the canister holder 226.

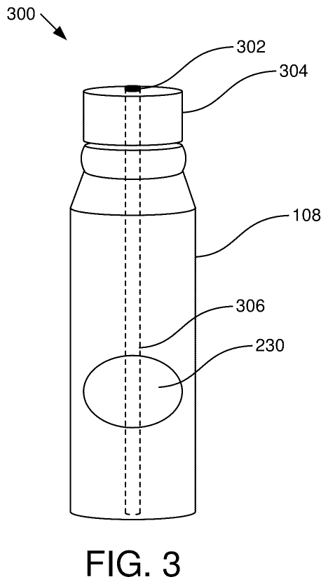

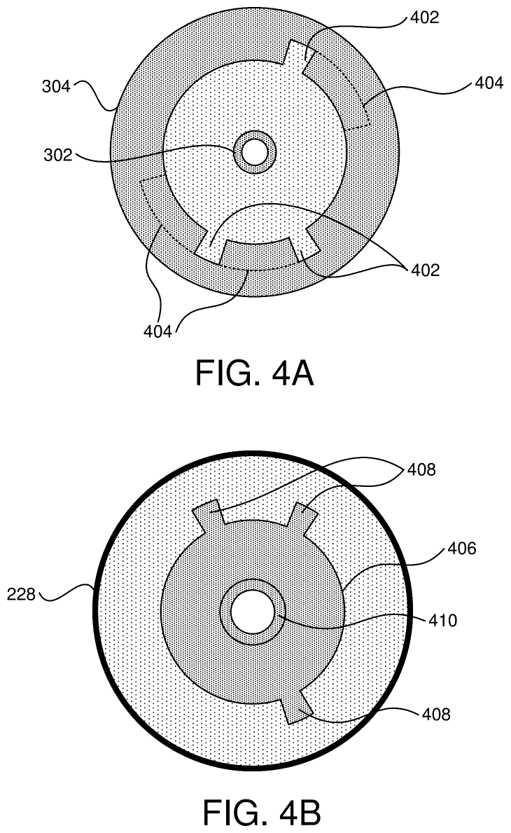

The receiver port 228, in some embodiments, includes a leak-tight seat that surrounds a canister valve of the keyed canister 108. The leak-tight seat, in some embodiments, allows the canister valve to move up and down while still maintaining a seal between the leak-tight seat and canister valve. The receiver port 228 includes keys that match with a keyed receiver top at the top of the keyed canister 108, which is described below with respect to the keyed canister 108 of FIGS. 3 and 4.

In some embodiments, the inhaler apparatus 100 includes a charging port 232 configured for a user to plug in a cord to recharge the rechargeable battery 218. In other embodiments, the charging port 232 is configured for communications in addition to battery charging. In some embodiments, the charging port 232 is a universal serial bus (“USB”) port, which allows both communications and battery charging. In some embodiments, the charging port 232 is a USB type C (“USB-C”) port configured for a USB-C cable. In other embodiments, the charging port 232 is configured for another type of port available now or in the future where the charging port 232 is configured for battery charging and/or communications. In some embodiments, the charging port 232 is different from a communications port (not shown) on the propellant control unit 102.

In some embodiments, the microcontroller 202, the lockout device 208, the wireless antenna 212, the charging port 232, and/or other electronics are encapsulated in an encapsulating material, such as epoxy, a potting compound, or other substance and sealed inside the propellant control unit 102 to prevent tampering. In some embodiments, the lockout device 208 is covered with a cylinder or other device to prevent the encapsulating material from interfering with operation of the lockout device 208.

In some embodiments, the inhaler apparatus 100 includes a nozzle 234 located below or within the mouthpiece 104 configured to aerosolize the inhalation substance from the canister valve of the keyed canister 108. Typically, the nozzle 234 is used when the inhalation substance is non-water based, such as an alcohol-based substance. In other embodiments, the nozzle 234 is replaced by a nebulizer, which is discussed above for water-based inhalation substances.

FIG. 3 is a front view 300 of a keyed canister 108 with a memory chip 230, according to various embodiments. the keyed canister 108 includes a canister valve 302 configured to release the inhalation substance stored in the keyed canister 108 when the canister valve 302 is depressed. The canister valve 302 is surrounded by a keyed receiver top 304. The keyed receiver top 304 includes recesses for a key shape of the receiver port 228. In some embodiments, the keyed receiver top 304 includes slots for the keys of the receiver port 228 so that when the keyed canister 108 is rotated after being inserted into the receiver port 228, the keys of the receiver port 228 extend into the slots to lock the keyed canister 108 into the receiver port 228.

The keyed canister 108 also includes a dip tube 306 in fluid communication with the canister valve 302 to an interior of the bottom of the keyed canister 108. The inhaler apparatus 100 is intended to be used upright as depicted in FIGS. 1A, 1B, and 2A so that the keyed canister 108 is also oriented with the keyed receiver top 304 is facing upward. In other dispensers, the canister is inverted so that liquid in the canister is at the canister valve. The dip tube 306 compensates for the keyed canister 108 being oriented with the keyed receiver top 304 facing upwards. The inhalation substance at the bottom of the keyed canister 108 is drawn into the dip tube 306 and out the canister valve 302. The keyed canister 108 also includes the memory chip 230, as depicted.

FIG. 4A is a top view of a keyed receiver top 304 of the keyed canister 108 of FIG. 3, according to various embodiments. FIG. 4B is a bottom view of a receiver port 228 in the inhaler apparatus of FIG. 1 depicting a keyed shape to receive the keyed receiver top 304 of FIG. 4A, according to various embodiments. The keyed canister 108 includes a canister valve 302 in the center of the keyed receiver top 304 of the keyed canister 108. The keyed receiver top 304 includes key slots 402 for the keys 408 of the receiver port 228.

In the embodiments of FIGS. 4A and 4B, the keys 408 are spaced around a circular center portion 406 of the receiver port 228 and align with the key slots 402 of the keyed receiver top 304 to allow a user to move the keyed canister 108 vertically and rotate the keyed canister 108 so the keys 408 align with the key slots 402 and then to press the keyed canister 108 up while the keys 408 move vertically through the key slots 402 to an end point. While at the end point, the keys 408 of the receiver port 228 are configured to slide into slots 404 of the keyed receiver top 304 while the user rotates the keyed canister 108 with respect to the propellant control unit 102.

The receiver port 228 includes a circular center portion 406 that fits into a recess of the keyed receiver top 304. The circular center portion 406 includes a tube receiver 410 that tightly fits and seals the canister valve 302 on the keyed canister 108. The canister valve 302 is able to slide within the tube receiver 410 so that depressing the activation button 110 after activation of the unlocked state dispenses a metered dose of the inhalation substance in the keyed canister 108. While a particular arrangement of keys 408 and corresponding key slots 402 is depicted in FIGS. 4A and 4B, many other combinations of the number of keys 408 and arrangement of the keys 408, and a diameter of the circular center portion 406 of the receiver port 228 are possible. In addition, other keying schemes are also contemplated herein, such as keys 408 that fit into slots similar to a screw or bolt, multiple layers of vertical key slots 402 and slots 404, and the like. In other embodiments, the keyed shape of the receiver port 228 does not include the circular center portion 406 and instead has a different shape. One of skill in the art will recognize one or more of the many ways to construct a keyed shape of the receiver port 228 and corresponding shape of the keyed receiver top 304.

In some embodiments, the keyed receiver top 304 is configured to lock the canister valve 302 until the canister 108 is inserted into the receiver port 228. In some examples, the keyed receiver top 304 includes a plate that is inserted into a notch in the canister valve 302 that holds the canister valve 302 from being depressed and rotating the canister 108 in the receiver port 228 moves the plate from the notch allowing the canister valve 302 to be depressed. In other embodiments, the keyed receiver top 304 includes a plate that covers the canister valve 302 and rotating the canister 108 within the receiver port 228 moves the plate so that the canister valve 302 is accessible. One of skill in the art will recognize other ways that the keyed receiver top 304 prevents the canister 108 from dispensing a dose of the inhalation substance until the canister 108 is fully inserted into the receiver port 228 of the dispenser 102.

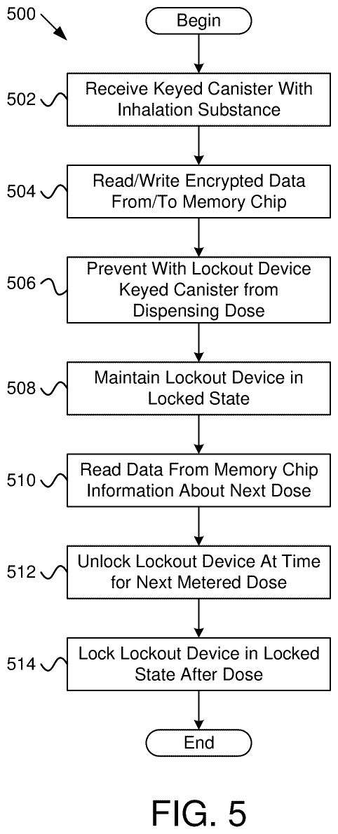

FIG. 5 is a schematic flowchart diagram illustrating a method for using an inhaler apparatus 100 with a prescription-controlled, metered-dose, aerosol inhaler device, according to various embodiments. The method 500 begins and receives 502, via a receiver port 228, a keyed canister 108 that includes an inhalation substance. The receiver port 228 includes a key shape to receive a keyed receiver top 304 of the keyed canister 108 and to reject canisters without the keyed receiver top 304. The method 500 reads 504, via a wireless data module 204, encrypted data from and writes 504 encrypted data to a memory chip 230 affixed to a side of the keyed canister 108 locked into the receiver port 228.

The method 500 prevents 506, using a lockout device 208 in a locked state, the keyed canister 108 from dispensing a metered dose of the inhalation substance while in a locked position. The method 500 maintains 508 the lockout device 208 in the locked state and reads 510 data from the memory chip 230 that includes information including timing for a next metered dose of the inhalation substance. The method 500 unlocks 512 the lockout device 208 at a time for the next metered dose of the inhalation substance, and locks 514 the lockout device 208 in the locked state in response to a user dispensing the next metered dose, and the method 500 ends. In various embodiments, all or a portion of the method 500 is implemented using the mouthpiece 104, the trigger button, the keyed canister 108, the activation button 110, the wireless data module 204, the lockout device module 206, the lockout device 208, the encryption module 210, the wireless antenna 212, the dispenser module 214, the rod 222, the notch 224, the receiver port 228, and/or the memory chip 230.

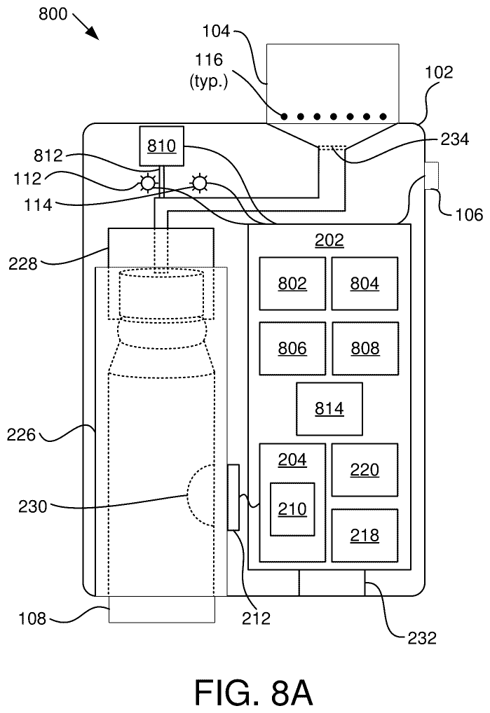

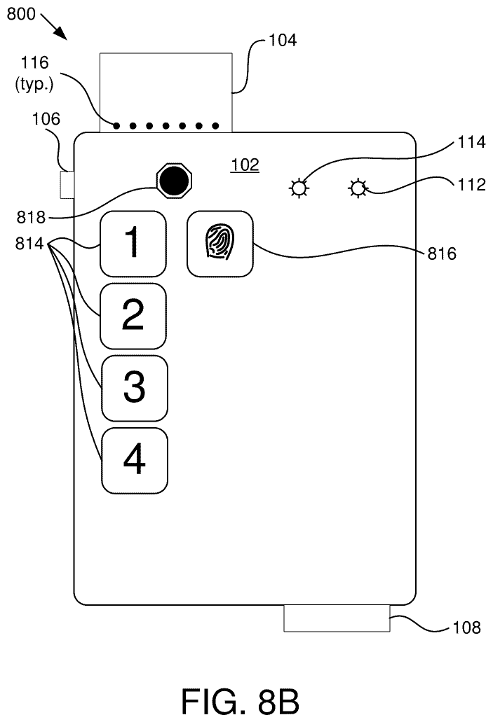

FIG. 8A is a front view illustrating yet another inhaler apparatus 800 with a prescription-controlled, metered-dose, aerosol inhaler device locking a canister 108 with a dispenser 102 and a user to the canister 108, according to various embodiments. FIG. 8B is a front view illustrating the inhaler apparatus 800 of FIG. 8A with a cover, according to various embodiments. Except as described below, elements of the inhaler apparatus 800 are substantially similar to the inhaler apparatus 100 of FIGS. 1A-1B and 2A-2E, such as the propellant control unit 102 (e.g., the dispenser 102, note that dispenser 102 and propellant control unit 102 are used interchangeably herein), the mouthpiece 104, the trigger button 106, the canister 108, the dose timer indicator 112, the dose ready indicator 114, the holes 116, the microcontroller 202, the wireless data module 204, the encryption module 210, the wireless antenna 212, the rechargeable battery 218, the voltage converter 220, the canister holder 226, the receiver port 228, the memory chip 230, the charging port 232 and the nozzle 234.

The inhaler apparatus 800 of FIGS. 8A and 8B differs from the inhaler apparatus 100 of FIGS. 1A, 1B, and 2A-2E in that there is no activation button 110 that a user presses to depress a rod 222, which would press on a canister valve 302 to dispense an inhalation substance from the dispenser 102. Instead, an internal actuator 810 receives a signal from the microcontroller 202 when dispensing is enabled. The internal actuator 810 then moves a rod 812 that presses the cannister valve 302 (not shown) which dispenses a dose of the inhalation substance when conditions are right. Having the internal actuator 810 prevents users from pressing an activation button 110 too hard, which may break the activation button 110 as the user is trying to get a regular dose or is trying to dispense a dose when the time for a dose has not be reached yet.

The inhaler apparatus 800 of FIGS. 8A and 8B includes a canister code module 802, a dispenser module 804, an identity input module 806, an ID comparison module 808, and a dose dispenser module 814, which are explained below. In various embodiments, the modules 802, 804, 806, 808 of FIG. 8A, in some embodiments, are implemented using executable code that is executed by the microcontroller 202. In other embodiments, all or a portion of the modules 802, 804, 806, 808 are implemented using a programmable hardware device, such as when the microcontroller 202 is implemented using an FPGA, a programmable logic array, etc. and/or implemented using hardware circuits.

The inhaler apparatus 800 includes a receiver port 228 within a dispenser 102 where the receiver port 228 is configured to receive a canister 108 that includes an inhalation substance. The dispenser 102 is configured to dispense an inhalation substance from the canister 108. In some embodiments, the canister 108 is a keyed canister 108 as described above and, in various embodiments, includes keys 408 that fit into keyed slots 402 of the receiver port 228. In other embodiments, the canister 108 may include slots, locking mechanisms, etc. where the keys 408, slots, locking mechanisms, etc. work together with keyed slots 402, keys, locking mechanisms, etc. of the receiver port 228 so that the canister 108 interlocks in the dispenser 102. In other embodiments, the canister 108 does not include keys 408 or other locking mechanisms and is sized to fit into the receiver port 228.

The inhaler apparatus 800 includes a wireless data module 204 configured to read encrypted data from and write encrypted data to a memory chip 230 affixed to the canister 108. The wireless data module 204 is described in detail above and serves to interact with the memory chip 230 to read and store dosage information, timing of doses of the inhalation substance, number of remaining doses, dates, information about a dispensing pharmacy, and other information useful to control dispensing of doses of the inhalation substance in the canister 108.

In some embodiments, the inhaler apparatus 800 includes a canister code module 802 configured, in response to data read from the memory chip 230 not including a cannister code, to write a cannister code to the memory chip 230 on the canister 108 where the cannister code is specific to the dispenser 102. In some embodiments, the canister code module 802 generates the canister code. In other embodiments, the canister code module 802 receives a canister code from another sources. In some examples, the canister code is written to the canister code module 802 by a pharmacist, a manufacturer of the dispenser 102, by a doctor, or other person. In some embodiments, a hash routine, an encryption routine, or the like is used to create the canister code before being written to the dispenser 102. In other embodiments, a canister code is written to the dispenser 102 and is transformed by the canister code module 802.

The purpose of the canister code is to pair the dispenser 102 with the canister 108 so that the canister 108 cannot be used in another dispenser 102, which helps to prevent a user from giving the canister 108 to another user, sharing the canister 108, etc. In some embodiments, the canister code is a hash or other unique code generated by the canister code module 802. In some embodiments, the canister code module 802 uses information about the dispenser 102, such as a serial number to create the canister code. In various embodiments, the canister code is unique and differs from all other canister codes.

In some embodiments, the canister code is encrypted by the encryption module 210 before being written to the memory chip 230 by the wireless data module 204. In such embodiments, the wireless data module 204 writes an encrypted canister code to the memory chip 230 of the canister 108. In some embodiments, the encryption module 210 includes an encryption key specific to dispensers 102 that are configured to receive the canister 108. In some cases, the encryption module 210 uses symmetric encryption to generate an encryption key that is distributed to dispensers 102 configured to receive the canister 108 and other similar canisters 108. In other embodiments, the encryption key is generated using asymmetric encryption and the encryption module 210 includes a private encryption key for decrypting an encrypted canister code and/or a public encryption key used to encrypt the canister code. In other embodiments, the encryption module 210 uses other encryption techniques.

In some embodiments, the inhaler apparatus 800 includes a dispenser module 804 configured to enable the dispenser 102 to dispense the inhalation substance from the canister 108 in response to determining that the memory chip 230 includes the cannister code and configured to lock out the dispenser 102 from dispensing the inhalation substance from the canister 108 in response to determining that the canister 108 does not have a cannister code or determining that the canister 108 has a different cannister code. The different canister code is for a different dispenser 102 and/or does not match the canister code that the canister code module 802 generated, received, etc. Thus, a canister 108 with a different canister code is unable to be used in the dispenser 102 and only canisters 108 that have been written with the canister code provided by the canister code module 802 of the dispenser 102 are able to be used to dispense a dose of the inhalation substance in the canister 108.

The inhaler apparatus 800, in some embodiments, includes an identity input module 806 configured to receive user identity (“ID”) information from a user. In some embodiments, the dispenser 102 includes a keypad and the user ID information from the user is a numerical code input by the user. In some embodiments, the keypad includes four buttons 814. The buttons 814, in some embodiments, are labeled 1-4, as depicted in FIG. 8B. In other embodiments, the buttons 814 are labeled A, B, C, and D. In other embodiments, the buttons 814 are labeled in some other manner. In other embodiments, the dispenser 102 includes more than four keys, such as 6 keys, 8 keys, etc. and the buttons 814 are labeled appropriately.

In other embodiments, the dispenser 102 includes a fingerprint reader 816 and the user ID information received by the identity input module 806 is a fingerprint of the user. In other embodiments, the dispenser 102 includes a camera 818 and the user ID information received by the identity input module 806 is an image of the user. In other embodiments, the dispenser 102 includes a retina scanner and the user ID information is a retina scan of the user. In other embodiments, the dispenser 102 includes another user input device that enables the user to input user ID information.

In the embodiments, the inhaler apparatus 800 includes an ID comparison module 808 configured to compare the user ID information with stored ID information. The stored ID information of the user stored in some embodiments on the memory chip 230 of the canister 108. In other embodiments, the stored ID information is stored on the dispenser 102. Where the stored ID information is stored on the canister 108, the canister 108 may be used with another dispenser 102. Where the stored ID information is stored on the dispenser 102, the user may only be able to use the dispenser 102.

In some embodiments, a dispensing authority stores the stored ID information of the user on the memory chip 230 of the canister 108 when the canister 108 is dispensed to the user and/or on the dispenser 102 when the dispenser 102 is provided to the user. In various embodiments, the dispensing authority is a pharmacist, a doctor, or other entity that dispenses canisters 108 and/or dispensers 102.

In the embodiments, the dispenser module 804 is configured to enable the dispenser 102 to dispense the inhalation substance from the canister 108 in response to the ID comparison module 808 determining that the user ID information matches the stored ID information and configured to lock out the dispenser 102 from dispensing the inhalation substance from the canister 108 in response to the ID comparison module 808 determining that the user ID information does not match the stored ID information.

In some embodiments, the inhaler apparatus 800 includes the canister code module 802, the dispenser module 804, the identity input module 806, and the ID comparison module 808. In such embodiments, the dispenser module 804 is configured to enable the dispenser 102 to dispense the inhalation substance from the canister 108 in response to determining that the memory chip 230 includes the cannister code and in response to the ID comparison module 808 determining that the user ID information matches the stored ID information and is configured to lock out the dispenser 102 from dispensing the inhalation substance from the canister 108 in response to one or more of determining that the canister 108 does not have a cannister code, determining that the canister 108 includes a different cannister code, and the ID comparison module 808 determining that the user ID information does not match the stored ID information.

In some embodiments, the dispenser 102 is configured with an internal actuator 810 configured to dispense the inhalation substance from the canister 108. In such embodiments, the inhaler apparatus 800 lacks a mechanical button that the user presses to mechanically press on the canister valve 302. Instead, the internal actuator 810 is configured to receive a signal from the microcontroller 202 to initiate dispensing of the inhalation substance from the canister 108. Having the internal actuator 810 provides a system where the user does not have a button to press and possibly to break when trying to dispense a dose of the inhalation substance during a regular dispensing time or between scheduled doses.

In some embodiments, the internal actuator 810 is a gear motor. In other embodiments, the internal actuator 810 is a solenoid configured to press the canister valve 302. In other embodiments, the internal actuator 810 is a motor configured to turn a gear and a rod 812 with teeth is then pressed into the canister valve 302. In other embodiments, the internal actuator 810 is another device capable of pressing on the canister valve 302 to dispense a dose of the inhalation substance from the canister 108.

In embodiments with an internal actuator 810, the inhaler apparatus 800 includes a dose dispenser module 814 configured to signal the internal actuator 810 to dispense a dose of the inhalation substance from the canister 108 in response to the dispenser module 804 enabling the dispenser 102 to dispense the inhalation substance. The dose dispenser module 814 requires no further action from the user.

In other embodiments, the microcontroller 202 includes the canister code module 802, the dispenser module 804, the identity input module 806, and the ID comparison module 808 and the dispenser 102 includes the lockout device module 206, the lockout device 208, the dispenser module 214, and/or the lockout failure module 216 and the dispenser 102 includes an activation button 110, a rod 222 with a notch 224, etc. as depicted in the inhaler apparatus 100 of FIGS. 1A, 1B, and 2A-2E. In such embodiments, the dispenser module 804 controls whether or not a dose is enabled or locked out and when a dose is enabled, the user then presses the activation button 110.

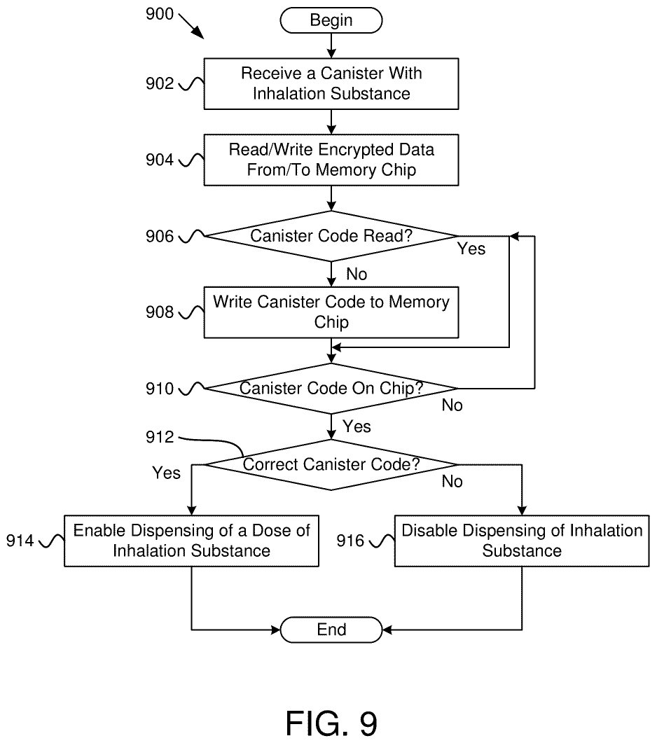

FIG. 9 is a schematic flowchart diagram illustrating a method 900 for using an inhaler apparatus 800 with a prescription-controlled, metered-dose, aerosol inhaler device and writing a unique canister code to a memory chip 230 of the canister 108, according to various embodiments. The method 900 begins and reads 902 a canister 108 with an inhalation substance. The canister 108 is received into a receiver port 228 of the dispenser 102 and the dispenser 102 is configured to dispense the inhalation substance from the canister 108 to a user. The canister 108 includes a memory chip 230 and the dispenser 102 includes a wireless data module 204 configured to read encrypted data from and write encrypted data to a memory chip 230 affixed to the canister 108.

The method 900 reads 904 encrypted data to and writes 904 encrypted data from a memory chip 230 affixed to the canister 108 when the canister 108 is inserted into the receiver port 228. The method 900 determines 906 whether a canister code is read from the memory chip 230. If the method 900 determines 906 that no canister code was read from the memory chip 230, the method 900 writes 908 a canister code to the memory chip 230 where the canister code is unique to the dispenser 102. If the method 900 determines 906 that a canister code was read from the memory chip 230, the method 900 does not write another canister code to the memory chip 230.

In subsequent operations, such as at a time for dispensing a dose of the inhalation substance, the method 900 determines 910 if there is a canister code on the memory chip 230. If the method 900 determines 910 that there is no canister code on the memory chip 230 of the canister 108, the method 900 returns and writes 908 a canister code to the memory chip 230. If the method 900 determines 910 that there is a canister code onto the memory chip 230, the method 900 determines 912 if the canister code is a correct canister code. The method 900, in some embodiments, determines 912 if the canister code is correct by comparing the canister code from the memory chip 230 with a canister code of the dispenser 102. If the method 900 determines 912 that the canister code is the correct canister code, the method 900 enables 914 dispensing of a dose of the inhalation substance, and the method 900 ends. If the method 900 determines that the canister code is incorrect, the method 900 disables 916 dispensing of inhalation substance from the canister 108, and the method 900 ends. In various embodiments, all or a portion of the method is implemented by the canister code module 802, the dispenser module 804, and/or the wireless data module 204.

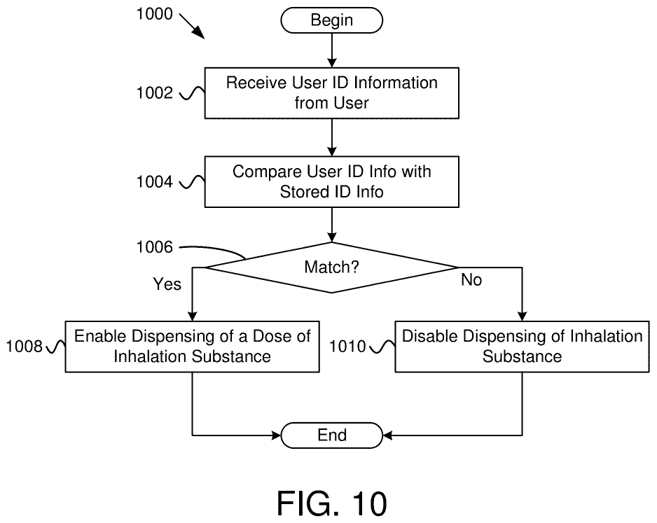

FIG. 10 is a schematic flowchart diagram illustrating a method 1000 for using an inhaler apparatus 800 with a prescription-controlled, metered-dose, aerosol inhaler device in comparing a user ID with a stored user ID and enabling dispensing in case of a match, according to various embodiments. The method 1000 begins and receives 1002 user ID information from a user where the user ID information is received at a dispenser 102 with a receiver port 228 configured to receive a canister 108 with an inhalation substance. The dispenser 102 is configured to dispense the inhalation substance from the canister 108 and is configured with a wireless data module configured to read encrypted data from and write encrypted data to a memory chip 230 affixed to the canister 108.

The method 1000 compares 1004 the user ID information with stored ID information. The stored ID information of the user is stored on the dispenser 102 and/or the memory chip 230 of the canister 108. The method 1000 determines 1006 if there is a match between the user ID information and the stored ID information. If the method 1000 determines 1006 that there is a match between the user ID information and the stored ID information, the method 1000 enables 1008 the dispenser 102 to dispense the inhalation substance from the canister 108, and the method 1000 ends. If the method 1000 determines 1006 that the user ID information does not match the stored ID information, the method 1000 disables 1010 the dispenser 102 from dispensing the inhalation substance from the canister 108, and the method 1000 ends. In various embodiments, all or a portion of the method 1000 is implemented using the wireless data module 204, the identity input module 806, the ID comparison module 808, and/or the dispenser module 804.

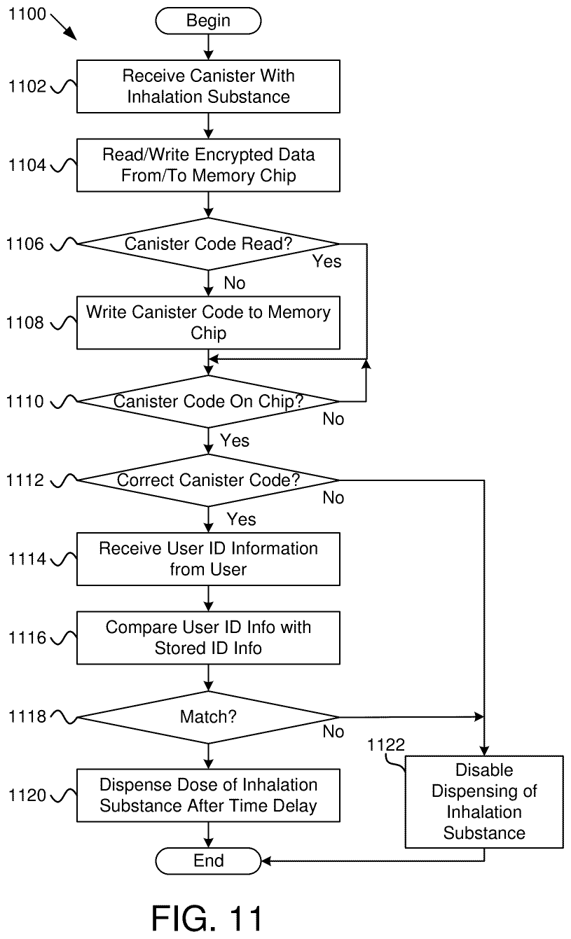

FIG. 11 is a schematic flowchart diagram illustrating a method 1100 for using an inhaler apparatus 800 with a prescription-controlled, metered-dose, aerosol inhaler device for dispensing a dose of an inhalation substance in response to a correct canister code being read from a memory chip 230 on a canister 108 and in response to a user ID matching a stored user ID, according to various embodiments. The method 1100 begins and receives 1102 a canister 108 with an inhalation substance into a receiver port 228 of a dispenser 102. The dispenser 102 is configured to dispense a dose of the inhalation substance from the canister 108.

The method 1100 reads 1104 encrypted data from and writes 1104 encrypted data to a memory chip 230 affixed to the canister 108 and determines 1106 if a canister code was read from the memory chip 230. If the method 1100 determines 1106 that a canister code was not read from the memory chip 230, the method 1100 writes 1108 a canister code to the memory chip 230. If the method 1100 determines 1106 that there is a canister code on the memory chip 230, the method 1100 does not write another canister code to the memory chip 230.

In subsequent operations, such as at a time for dispensing a dose of the inhalation substance, the method 1100 determines 1110 if there is a canister code on the memory chip 230 of the canister 108. If the method 1100 determines 1110 that there is no canister code on the memory chip 230 of the canister 108, the method 1100 returns and writes 1108 a canister code to the memory chip 230. If the method 1100 determines 1110 that there is a canister code on the memory chip 230, the method 1100 determines 1112 if the canister code is correct. If the method 1100 determines 1112 that the canister code is correct, the method 1100 receives 1114 user ID information from a user and compares 1116 the user ID information with stored ID information.

The method 1100 determines 1118 if the user ID information matches the stored ID information. If the method 1100 determines 1118 that the user ID information matches the stored ID information, the method 1100 dispenses 1120 a dose of the inhalation substance from the canister 108 after a time delay, and the method 1100 ends. If the method 1100 determines 1112 that the canister code is incorrect or if the method 1100 determines 1118 that the user ID information does not match the stored ID information, the method 1100 disables 1122 dispensing of inhalation substance from the canister 108, and the method 1100 ends. In various embodiments, all or a portion of the method 1100 is implemented using the canister code module 802, the dispenser module 804, the wireless data module 204, the identity input module 806, and/or the ID comparison module 808.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An inhaler apparatus comprising:
a receiver port of a dispenser, the receiver port configured to receive a canister comprising an inhalation substance, wherein the dispenser is configured to dispense the inhalation substance from the canister;
a wireless data module configured to read encrypted data from and write encrypted data to a memory chip affixed to the canister;
a canister code module configured, in response to data read from the memory chip not comprising a cannister code, to write a cannister code to the memory chip on the canister, wherein the cannister code is specific to the dispenser; and
a dispenser module configured to enable the dispenser to dispense the inhalation substance from the canister in response to determining that the memory chip comprises the cannister code and configured to lock out the dispenser from dispensing the inhalation substance from the canister in response to one of: determining that the canister does not have a cannister code and determining that the canister comprises a different cannister code,
wherein at least a portion of said modules comprise one or more of hardware circuits, programmable hardware devices and executable code, the executable code stored on one or more computer readable storage media.

2. The inhaler apparatus of claim 1, further comprising an encryption module configured to encrypt the canister code prior to the canister code module writing the canister code to the memory chip of the canister, wherein the encrypted canister code is stored on the memory chip.

3. The inhaler apparatus of claim 2, wherein the encryption module comprises an encryption key specific to dispensers configured to receive the canister.

4. The inhaler apparatus of claim 3, wherein the encryption key is generated using one of symmetric encryption and the encryption module comprises a common encryption key and asymmetric encryption and the encryption module comprises a private encryption key.

5. The inhaler apparatus of claim 1, further comprising:
an identity input module configured to receive user identity (“ID”) information from a user; and
an ID comparison module configured to compare the user ID information with stored ID information, the stored ID information of the user stored on the dispenser and/or the memory chip of the canister,
wherein the dispenser module is configured to enable the dispenser to dispense the inhalation substance from the canister in response to determining that the memory chip comprises the cannister code and in response to the ID comparison module determining that the user ID information matches the stored ID information and configured to lock out the dispenser from dispensing the inhalation substance from the canister in response to one or more of:
determining that the canister does not have a cannister code;
determining that the canister comprises a different cannister code; and
the ID comparison module determining that the user ID information does not match the stored ID information.

6. The inhaler apparatus of claim 5, wherein the identity input module receives the ID information from the user via:
a keypad on the dispenser, wherein the user ID information comprises a code input by the user via the keypad;
a fingerprint reader on the dispenser, wherein the user ID information comprises a fingerprint of the user;
a camera on the dispenser, wherein the user ID information comprises an image of the user; and/or
a retina scanner on the dispenser, wherein the user ID information comprises a retina scan of the user.

7. The inhaler apparatus of claim 5, wherein a dispensing authority stores the stored ID information of the user on the memory chip when the canister is dispensed to the user and/or on the dispenser when the dispenser is provided to the user.

8. The inhaler apparatus of claim 5, further comprising a dose dispenser module configured to signal an internal actuator to dispense a dose of the inhalation substance from the canister in response to the dispenser module enabling the dispenser to dispense the inhalation substance, wherein the dose dispenser module requires no further action from the user.

9. The inhaler apparatus of claim 8, wherein the dose dispenser module dispenses the dose after a time delay.

10. The inhaler apparatus of claim 1, wherein the memory chip comprises one of:
a read/write radio frequency identifier (“RFID”) chip;
a memory chip readable and writable using near-field communication (“NFC”); and
a memory chip readable and writable using Bluetooth low energy (“BLE”) communications.

11. An inhaler apparatus comprising:
a receiver port of a dispenser, the receiver port configured to receive a canister comprising an inhalation substance, wherein the dispenser is configured to dispense the inhalation substance from the canister;
a wireless data module configured to read encrypted data from and write encrypted data to a memory chip affixed to the canister;
an identity input module configured to receive user identity (“ID”) information from a user;
an ID comparison module configured to compare the user ID information with stored ID information, the stored ID information of the user stored on the dispenser and/or the memory chip of the canister; and
a dispenser module is configured to enable the dispenser to dispense the inhalation substance from the canister in response to the ID comparison module determining that the user ID information matches the stored ID information and configured to lock out the dispenser from dispensing the inhalation substance from the canister in response to the ID comparison module determining that the user ID information does not match the stored ID information,
wherein at least a portion of said modules comprise one or more of hardware circuits, programmable hardware devices and executable code, the executable code stored on one or more computer readable storage media.

12. The inhaler apparatus of claim 11, further comprising:
a canister code module configured, in response to data read from the memory chip not comprising a cannister code, to write a cannister code to the memory chip on the canister, wherein the cannister code is specific to the dispenser,
wherein the dispenser module is configured to enable the dispenser to dispense the inhalation substance from the canister in response to determining that the memory chip comprises the cannister code and in response to the ID comparison module determining that the user ID information matches the stored ID information and configured to lock out the dispenser from dispensing the inhalation substance from the canister in response to one or more of:
determining that the canister does not have a cannister code;
determining that the canister comprises a different cannister code; and
the ID comparison module determining that the user ID information does not match the stored ID information.

13. The inhaler apparatus of claim 12, further comprising an encryption module configured to encrypt the canister code prior to the canister code module writing the canister code to the memory chip of the canister, wherein the encrypted canister code is stored on the memory chip.

14. The inhaler apparatus of claim 13, wherein the encryption module comprises an encryption key specific to dispensers configured to receive the canister.

15. The inhaler apparatus of claim 11, wherein the identity input module receives the ID information from the user via:
a keypad on the dispenser, wherein the user ID information comprises a code input by the user via the keypad;
a fingerprint reader on the dispenser, wherein the user ID information comprises a fingerprint of the user;
a camera on the dispenser, wherein the user ID information comprises an image of the user; and/or
a retina scanner on the dispenser, wherein the user ID information comprises a retina scan of the user.

16. The inhaler apparatus of claim 11, wherein the stored ID information is stored on the memory chip of the canister and wherein a dispensing authority stores the stored ID information of the user on the memory chip when the canister is dispensed to the user.

17. The inhaler apparatus of claim 11, further comprising a dose dispenser module configured to signal an internal actuator to dispense a dose of the inhalation substance from the canister in response to the dispenser module enabling the dispenser to dispense the inhalation substance, wherein the dose dispenser module requires no further action from the user.

18. The inhaler apparatus of claim 17, wherein the dose dispenser module dispenses the dose after a time delay.

19. An inhaler apparatus comprising:
a receiver port of a dispenser, the receiver port configured to receive a canister comprising an inhalation substance, wherein the dispenser is configured to dispense the inhalation substance from the canister;
a wireless data module configured to read encrypted data from and write encrypted data to a memory chip affixed to the canister;
a canister code module configured, in response to data read from the memory chip not comprising a cannister code, to write a cannister code to the memory chip on the canister, wherein the cannister code is specific to the dispenser;
an identity input module configured to receive user identity (“ID”) information from a user; and
an ID comparison module configured to compare the user ID information with stored ID information, the stored ID information of the user stored on the dispenser and/or the memory chip of the canister; and
a dispenser module configured to enable the dispenser to dispense the inhalation substance from the canister in response to determining that the memory chip comprises the cannister code and in response to the ID comparison module determining that the user ID information matches the stored ID information and configured to lock out the dispenser from dispensing the inhalation substance from the canister in response to determining that the canister does not have a cannister code, in response to determining that the canister comprises a different cannister code, and in response to the ID comparison module determining that the user ID information does not match the stored ID information,
wherein at least a portion of said modules comprise one or more of hardware circuits, programmable hardware devices and executable code, the executable code stored on one or more computer readable storage media.

20. The inhaler apparatus of claim 19, wherein the identity input module receives the ID information from the user via:
a keypad on the dispenser, wherein the user ID information comprises a code input by the user via the keypad;
a fingerprint reader on the dispenser, wherein the user ID information comprises a fingerprint of the user;
a camera on the dispenser, wherein the user ID information comprises an image of the user; and/or a retina scanner on the dispenser, wherein the user ID information comprises a retina scan of the user.
